(19) United States
(12) Patent Application Publication
(10) Pub. No.: US 2014/0234325 A1
(43) Pub. Date: Aug. 21, 2014

(54) ADJUVANT THERAPY FOR STAPHYLOCOCCAL INFECTION WITH ENTEROTOXIN SPECIFIC MABS

(71) Applicant: ALBERT EINSTEIN COLLEGE OF MEDICINE OF YESHIVA UNIVERSITY, Bronx, NY (US)

(72) Inventors: Bettina Fries, New Rochelle, NY (US); Avanish Varshney, Bronx, NY (US); Emily Cook, Exeter (GB); Xiaobo Wang, New York, NY (US)

(21) Appl. No.: 14/346,981

(22) PCT Filed: Sep. 26, 2012
(86) PCT No.: PCT/US12/57186

§ 371 Date: Mar. 25, 2014

Related U.S. Application Data
(60) Provisional application No. 61/539,689, filed on Sep. 27, 2011.

(51) Int. Cl. C07K 16/12

(52) U.S. Cl. CPC C07K 16/1271
USPC 424/139.1; 530/387.9; 530/387.3

(57) ABSTRACT

Antibodies to SEB, fragments thereof, and compositions comprising such are provided. Therapies for staphylococcal infection are provided, as well as assays for identifying additional agents useful in such therapies. An isolated antibody, or an isolated antigen-binding fragment of an antibody, is provided which antibody or antigen-binding fragment binds to staphylococcal enterotoxin B (SEB) and which antibody or antigen-binding fragment comprises a heavy chain variable CDR3 comprising the sequence RIYYGNNGGVMDY (SEQ ID NO:30); ARTAGLLAPMDY (SEQ ID NO:31); ARDTMRKCYCELKLKPPAEHPGPA (SEQ ID NO:32) or VRDL YGDYVGRY A Y (SEQ ID NO:48).

### CROSS-REFERENCE TO RELATED APPLICATIONS

This application claims benefit of U.S. Provisional Application No. 61/539,689, filed Sep. 27, 2011, the contents of which are hereby incorporated by reference.

### STATEMENT OF GOVERNMENT SUPPORT

This invention was made with government support under grant numbers U54-AI057158 and 5T32-AI007506 awarded by the National Institutes of Health and grant number W911NF0710053 awarded by the Department of Defense. The government has certain rights in the invention.

### BACKGROUND OF THE INVENTION

The disclosures of all publications referred to in this application are hereby incorporated by reference in their entirety into the subject application to more fully describe the art to which the subject invention pertains.

The Staphylococcal enterotoxins (SEs) comprise a family of distinct toxins (A-E) all of which are excreted by various strains of Staphylococcus aureus (S. aureus) (1). Staphylococcal enterotoxin B (SEB) is a well characterized 28 kDa protein that is related to SEC1-3 on the basis of sequence homology (1, 2). SEB is a superantigen that triggers cytokine production and T-cell proliferation by cross-linking MHC class II molecules on antigen presenting cells and T-cell receptors (TCR) (2-5). In humans, SEB can trigger toxic shock, profound hypotension and multi-organ failure. SEB is the major enterotoxin associated with non-menstrual toxic shock syndrome and accounts for the majority of intoxications that are not caused by toxic shock syndrome toxin 1 (TSST-1). In addition, some reports indicate that SEB induces an IgE response and thereby might contribute to the pathogenesis of asthma, chronic rhinitis, and dermatitis (6-9). SEB is considered a select agent. The quantities needed to produce a desired effect are much lower than with synthetic chemicals. Also SEB can be easily produced in large quantities (10).

Currently there are no therapies available for treating enterotoxin-induced shock, but clinical data suggests that immunoglobulins can alleviate disease (11). Moreover, passive administration of pooled human immunoglobulin, as well as murine and chicken antibodies (Abs) can protect against SEB induced lethal shock (SEBILS) in murine and primate animal models as well as against SEB triggered release of cytokines by SEB stimulated T-cells (12, 13). The efficacy of humoral immunity in protection against SEB was established by demonstrating an inverse relationship between susceptibility and antibody (Ab) titer (13-16) and protection in mice and non-human primates. Protection correlated with the titer of Ab to SEB (17-19). The C terminus of the protein has been proposed to be the predominant epitope recognized by human B-cells (20).

The present invention addresses this need and identifies a novel epitopes on SEB, and provides antibodies thereto and related therapies.

### SUMMARY OF THE INVENTION

An isolated antibody, or an isolated antigen-binding fragment of an antibody, is provided which antibody or antigen-binding fragment binds to staphylococcal enterotoxin B (SEB) and which antibody or antigen-binding fragment comprises a heavy chain variable CDR3 comprising the sequence RIYYGNNGGVMDY (SEQ ID NO:30); ARTAGLLAPMDY (SEQ ID NO:31); ARDTMRKCYCELKLKPPAEHPGPA (SEQ ID NO:32) or VRDLYGDYVGRYAY (SEQ ID NO:48).

Also provided is a composition comprising any of the antibodies and/or antigen-binding fragments described herein.

Also provided is an isolated antibody, or an isolated fragment of an antibody, which antibody or fragment (i) binds to staphylococcal enterotoxin B (SEB) comprising SEQ ID NO:1 and does not bind to the polypeptide set forth in SEQ ID NO:2, and (ii) recognizes residues 135, 137, 186, 235 and 236 of SEQ ID NO:1; residues 135, 137, 186, 188, 231, 233, 235 and 236 of SEQ ID NO:1; residues 135 and 186 of SEQ ID NO:1; or residues 135, 137, 186, 188, 235 and 236 of SEQ ID NO:1.

Also provided is an isolated antibody, or an isolated fragment of an antibody, which antibody or fragment binds to staphylococcal enterotoxin B (SEB) comprising SEQ ID NO:1 and which antibody or fragment

(i) exhibits reduced binding to a modified SEB compared to its binding to SEB comprising SEQ ID NO:1, wherein the modified SEB is modified relative to SEB comprising SEQ ID NO:1 by having any one or more of the residues numbered 135, 137, 186, 235 and 236 of SEQ ID NO:1 mutated to an alanine, but which does not exhibit reduced binding if the residue numbered 231 of SEQ ID NO:1 is mutated to an alanine; or
(ii) exhibits reduced binding to a modified SEB compared to its binding to SEB comprising SEQ ID NO:1, wherein the modified SEB is modified relative to SEB comprising SEQ ID NO:1 by having any one or more of the residues numbered 135 and 186 of SEQ ID NO:1 mutated to an alanine, but which does not exhibit reduced binding if the residue numbered 231 of SEQ ID NO:1 is mutated to an alanine;
(iii) exhibits reduced binding to a modified SEB compared to its binding to SEB comprising SEQ ID NO:1, wherein the modified SEB is modified relative to SEB comprising SEQ ID NO:1 by having any one or more of the residues numbered 135, 137, 186, 188, 235 and 236 of SEQ ID NO:1 mutated to an alanine, but which exhibits increased binding to a second modified SEB compared to binding to SEB comprising SEQ ID NO:1, wherein the second modified SEB is modified relative to SEQ ID NO:1 by having any one or more of the residues numbered 229, 233 or 231 of SEQ ID NO:1 mutated to an alanine; or
(iv) exhibits reduced binding to a modified SEB compared to its binding to SEB comprising SEQ ID NO:1, wherein the modified SEB is modified relative to SEB comprising SEQ ID NO:1 by having any one or more of the residues numbered 135, 137, 186, 235 and 236 of SEQ ID NO:1 mutated to an alanine, but which does not exhibit reduced binding if one or more of the residues numbered 188, 231 or 233 of SEQ ID NO:1 is mutated to an alanine.

Also provided is an isolated antibody or the antigen-binding fragment of an antibody of any of the described antibodies, wherein the antibody is a human antibody, a humanized antibody or a chimeric antibody. In an embodiment, the antibody is a monoclonal antibody. In an embodiment, the antibody is a human antibody. In an embodiment, the fragment is of a human antibody, of a humanized antibody or of a chimeric antibody. In an embodiment, the fragment is of a monoclonal antibody. In an embodiment, the fragment is of a human antibody. In an embodiment, the fragment comprises an Fab, an Fab′, an F(ab′)2, an Fd, an Fv, a complementarity determining region (CDR), or a single-chain antibody (scFv).

A composition is provided comprising any of the described isolated antibodies or the described isolated fragments of an antibody. A pharmaceutical composition is provided comprising any of the described isolated antibodies or the described isolated fragments of an antibody.

Also provided is a method of treating a disease associated with a staphylococcus infection in a subject having the disease, or preventing a disease associated with a staphylococcus infection in a subject at risk of the disease, comprising administering to the subject an amount of an antibody, or antigen-binding fragment thereof, directed to SEB as described herein, or an amount of an antibody or antigen-binding fragment thereof directed to a conformational epitope of staphylococcal enterotoxin B (SEB), effective to treat the disease. In an embodiment, the antibody is a monoclonal antibody. In an embodiment, the antigen binding fragment is a fragment of a monoclonal antibody. In an embodiment, the SEB comprises SEQ ID NO:1.

A method is provided of treating a disease associated with a staphylococcus infection in a subject having the disease, or of preventing a disease associated with a staphylococcus infection in a subject at risk thereof, comprising administering to the subject an amount of at least two different monoclonal antibodies or antigen-binding fragment thereof, wherein each monoclonal antibody is directed to staphylococcal enterotoxin B (SEB), effective to treat the disease. In an embodiment, the SEB comprises SEQ ID NO:1. In an embodiment, the monoclonal antibodies or antigen-binding fragment thereof each recognize a conformational epitope of SEB. In an embodiment, the monoclonal antibodies or antigen-binding fragments each do not bind to a modified staphylococcal enterotoxin B which is modified relative to SEB comprising SEQ ID NO:1 by not comprising the C-terminal ten amino acid residues of the SEQ ID NO:1.

In an embodiment of the methods described herein, the disease is sepsis, SEB-mediated shock, a staphylococcus aureus infection, bacteremia, or staphylococcus aureus-associated atopic dermatitis. In an embodiment, the disease is a staphylococcus aureus infection. In an embodiment, the disease is a staphylococcus aureus skin infection. In an embodiment, the disease is a staphylococcus aureus bacteremia. In an embodiment, the staphylococcus aureus is methicillin-resistant staphylococcus aureus. In an embodiment, the staphylococcus aureus is methicillin-sensitive staphylococcus aureus. In an embodiment, one antibody is neutralizing and the other antibody is not neutralizing. In an embodiment, both antibodies are neutralizing. In an embodiment, neither antibody alone is neutralizing. In an embodiment, at least one administered monoclonal antibody or antigen-binding fragment thereof is an antibody or antigen-binding fragment as described herein. In an embodiment, the antibody or antigen-binding fragment thereof is, or antibodies or antigen-binding fragments thereof are, administered prophylactically. In an embodiment, the antibody or antigen-binding fragment thereof is, or antibodies or antigen-binding fragments thereof are, administered after the disease has manifested.

Also provided is a method for identifying a candidate agent as an agent for treating a disease associated with a staphylococcus infection comprising contacting staphylococcal enterotoxin B (SEB) comprising SEQ ID NO:1 with the candidate agent and determining if the candidate agent binds to, or competes with an antibody binding to, residues 135, 137, 186, 235 and 236 of SEQ ID NO:1; residues 135, 137, 186, 188, 231, 233, 235 and 236 of SEQ ID NO:1; residues 135 and 186 of SEQ ID NO:1; or residues 135, 137, 186, 188, 235 and 236 of SEQ ID NO:1, wherein if the candidate agent binds to, or competes with the antibody binding to, residues 135, 137, 186, 235 and 236 of SEQ ID NO:1; residues 135, 137, 186, 188, 231, 233, 235 and 236 of SEQ ID NO:1; residues 135 and 186 of SEQ ID NO:1; or residues 135, 137, 186, 188, 235 and 236 of SEQ ID NO:1 then the candidate agent is identified as an agent for treating a disease associated with a staphylococcus infection and wherein if the candidate agent does not bind to, or does not compete with the antibody binding to, residues 135, 137, 186, 235 and 236 of SEQ ID NO:1; residues 135, 137, 186, 188, 231, 233, 235 and 236 of SEQ ID NO:1; residues 135 and 186 of SEQ ID NO:1; or residues 135, 137, 186, 188, 235 and 236 of SEQ ID NO:1, then the candidate agent is not identified as an agent for treating a disease associated with a staphylococcus infection.

An isolated antibody is provided which inhibits SEB-induced human T-cell proliferation and SEB-induced human T-cell IL-2 and IFN-γ production when bound to a human T-cell. In an embodiment, the antibody is a human antibody, a humanized antibody or a chimeric antibody. In an embodiment, the antibody is a monoclonal antibody. In an embodiment, the antibody is a human antibody. In an embodiment, the antibody is a humanized antibody.

Also provided is an isolated antibody, or the isolated antigen-binding fragment of an antibody, which antibody or antigen-binding fragment binds to staphylococcal enterotoxin B (SEB) comprising SEQ ID NO:1 and which antibody or antigen-binding fragment comprises an amino acid sequence comprising three CDRs, each one of which has at least 90% identity to a different heavy chain variable CDR selected from CDR1, CDR2 and CDR3 as set forth in SEQ ID NO:36, 39, and 30, or from CDR1, CDR2 and CDR3 as set forth in SEQ ID NO:37, 40, and 31, or from CDR1, CDR2 and CDR3 as set forth in SEQ ID NO:49, 50, and 48. In an embodiment, one, two or three of the CDRs, each have at least one of 91%, 92%, 93%, 94%, 95%, 96%, 97%, 98% or 99% identity to a different heavy chain variable CDR selected from CDR1, CDR2 and CDR3 as set forth in SEQ ID NO:36, 39, and 30, or from CDR1, CDR2 and CDR3 as set forth in SEQ ID NO:37, 40, and 31, or from CDR1, CDR2 and CDR3 as set forth in SEQ ID NO:49, 50, and 48.

Also provided is an isolated antibody, or the isolated antigen-binding fragment of an antibody, which antibody or antigen-binding fragment binds to staphylococcal enterotoxin B (SEB) comprising SEQ ID NO:1 and which antibody or antigen-binding fragment comprises an amino acid sequence comprising three CDRs, each one of which is at least 90%, or at least 95%, homologous to a different heavy chain variable CDR selected from CDR1, CDR2 and CDR3 as set forth in SEQ ID NO:18, 22, or 26.

Also provided is an isolated antibody, or an isolated antigen-binding fragment of an antibody, which antibody or antigen-binding fragment binds to staphylococcal enterotoxin B (SEB) and which comprises (a) two heavy chains each comprising the sequence set forth in SEQ ID NO:8, 22, or 26, and (b) two light chains each comprising the sequence set forth in SEQ ID NO:19, 23 and 27.

A composition is provided comprising any of the antibody or antigen-binding fragments described herein. In an embodiment, the composition is a pharmaceutical composition and comprises a pharmaceutically acceptable carrier.

Also provided is an antibody or antigen binding fragment of an antibody as described herein, for treating a staphylococcus aureus infection, staphylococcus aureus bacteremia, staphylococcus aureus-associated sepsis, SEB-mediated shock, or staphylococcus aureus-associated atopic dermatitis in a subject. In an embodiment, the disease is a staphylococcus aureus skin infection.

Also provided is an antibody, or antigen-binding fragment of an antibody, as described herein, for treating a disease associated with a staphylococcus infection in a subject, wherein the antibody or antibody fragment is administered concurrently, separately or sequentially with a second antibody or second antibody fragment directed against SEB, wherein the second antibody or second antibody fragment is of a different sequence than the antibody, or antigen-binding fragment of an antibody. In an embodiment, the second antibody or second antibody fragment is also as described herein. Also provided is a first antibody, or antigen-binding fragment thereof, and a second antibody, or antigen-binding fragment thereof, wherein the first and second antibody or fragments thereof are as described herein, and wherein the first and second antibody, or fragments thereof, have different sequences, as a combined preparation for treating a disease associated with a staphylococcus infection in a subject. Also provided is a first antibody, or antigen-binding fragment thereof, for use with a second antibody, or antigen-binding fragment thereof, wherein the first and second antibody or fragments thereof are as described herein and wherein the first and second antibody, or fragments thereof, have different sequences, for treating a disease associated with a staphylococcus infection in a subject. In an embodiment, the disease is a staphylococcus aureus infection, staphylococcus aureus bacteremia, staphylococcus aureus-associated sepsis, SEB-mediated shock, or staphylococcus aureus-associated atopic dermatitis. In an embodiment, the disease is a staphylococcus aureus skin infection.

### BRIEF DESCRIPTION OF THE DRAWINGS

FIG. 1: Western blot analysis of mAbs 20B1, 14G8, 6D3, and 4C7 shows specificity of mAbs for SEB and not for SEA and TSST.

FIG. 2: Schematic of SEB sequence in MRSA and MSSA strains demonstrate the additional nucleotide thymidine found in all MRSA strains at position 703 which results in 3-aa residues change in the C-terminal part of the protein.

FIG. 3A-3D: Inhibition of T-cell proliferation and cytokine production by treatment with SEB specific mAb 20B1, 14G8, 6D3, and 4C7 individually or in different combinations. A, SEB-induced T-cell proliferation was measured by ViaLight HS Cell Proliferation kit after 48 h (A) and 96 h (B) and inhibited in the presence of all three mAb except 4C7. IFNγ (C) and IL-2 (D) were measured by ELISA in the supernatant of SEB stimulated T-cells (n=3 wells per condition). Cytokines were significantly (p<0.05 by t test) lower in the presence of mAbs relative to conditions with no specific antibody. The bars represent the S.D. derived from triplicate wells from same experiment.

FIG. 4A-4D: Protection against SEBILS was tested in BALB/c and HLA-DR3 mice (n=10 per group) that were injected intraperitoneally with 20 μg of SEB for BALB/c (0 h) (A and B), or 50 μg of SEB for HLA-DR3 mice (0 and 48 h) (C and D). Analysis of survival data were performed using Mantel-Cox Test. In the BALB/c model mAb 20B1 was protective at doses of 500 μg (p<0.0001) as well as 100 μg (p=<0.0003). HLA DR-3 mice that were treated intraperitoneally with 500 μg 20B1 at the same time were 100% protected whereas all SEB-injected mice treated with PBS or up to 1000 μg of mAbs 14G8 or 6D3 (HLA/DR3) died within 6 days (p=<0.0001). In contrast, mice treated with combination of mAbs 6D3 and 14G8 survived although monotherapy with the individual mAb was not protective. Similar enhanced protection was observed in the BALB/c mouse model when 20B1 was combined either with 6D3 or 14G8. No enhanced protection was found when 4C7 was administered.

FIG. 5. Protection against MRSA-derived SEB protein induced lethal shock was also determined in BALB/c mice by treatment with mAb 20B1 (p=0.0109). n=10 each group. Analysis of survival data were performed using Mantel-Cox Test.

FIG. 6A-6B. SEB level in the serum of (A) BALB/c and (B) HLA-DR3 mice (n=10 per group) was measured by ELISA. Note that mice injected with SEB and mAb 20B1 exhibited the highest SEB serum levels both in BALB/c and HLA/DR3 mice. Bars are averages of SEB measurements in the serum of five mice in each group and brackets denote intra-assay SD. The experiment was repeated and yielded similar differences. Gala, galactosamine.

FIG. 7: Capture ELISA with mAbs shows that two different SEB-specific mAbs can bind to SEB at the same time. Bars represent the average of three absorbance units at wavelength 405 nm and brackets denote intra-assay S.D. Inset: schematic diagram of ELISA, which applies to this experiment.

FIG. 8A-8E: (A) schematic diagram of SEB deletion mutants. (B) SDS-PAGE shows the expression of SEB and deletion mutants (M, marker, 1, uninduced cells, 2, induced SEB, 3, induced mutant-1 (5del SEB), 4, induced mutant-2 (11 del SEB), 5, induced mutant-3 (15 del SEB). (C) Western blot with mAbs and SEB deletion mutants shows that all three mAbs fail to bind to mutant 2 (11 residue deletion) and 3 (15 residue deletion). Not shown is that these mAbs also do not bind to the shorter SEB fragments. (D) dot blot analysis shows binding of 10-mer peptide with all three mAbs with SEB and mutant-1 and no binding with mutant-2. The binding affinity for the 10-mer peptide was low. (E) ELISA with purified SEB mutants protein (1 and 2) confirmed no binding of mutant 2 by mAbs 20B1, 14G8, and 6D3. FL=full-length.

FIG. 9A-9D: ELISA shows the effect of binding using different site directed mutagenesis proteins. Mutant proteins were coated in polystyrene plates at a concentration of 0.5 μg/ml. Further mAb 20B1 or 14G8 or 6D3 or 4C7 was added, detected by alkaline phosphatase (AP)-conjugated goat anti-mouse IgG1 and developed by PNPP tablets. The x-axis represents absorbance at 405 nm and y-axis represents the log of antibody concentration (in μg). Results identify different critical residues, which could interact with the individual SEB specific mAbs. For mAb 20B1 mutation of residues 135-R, 137-F, 186-Y, 235 & 236-T affected binding. The residues 135-R, 186-Y were required for the interaction with mAb 6D3. mAb 14G8 bound to residues 135-R, 137-F, 186-Y, 188-K, 231-E, 233-Y, and 235, 236-T, whereas mAb 4C7 interacts with 135-R, 137-F, 186-Y, 188-K, and 235, 236-T.

FIG. 10A-10D: Schematic representation of the potential residues recognized by SEB specific mAbs 20B1, 14G8, 6D3, and 4C7. All mAbs recognize non-continuous residues that are likely to contribute to conformational epitopes. (A) schematic illustration of the three-dimensional structure of SEB recognizing potential residues of mAbs. (B) schematic diagram of expanded view of the β-sheet formed by the three strands, which could disrupt by deleting C-terminal residues. (C) surface plot of SEB shows mutated residues (dark gray color) which are distinct from (D) the MHC surface (rotating 180 degrees around vertical axis) shown in lightest gray (residues 43, 44, 45, 46, 47, 65, 67, 89, 92, 94, 96, 98, 115, 209, 211, 215) and TCR surface in light gray (residues 18, 19, 20, 22, 23, 26, 60, 90, 91, 177, 178, and 210).

FIG. 11: Protection against MSSA derived SEB protein induced lethal shock was also determined in BALB/c mice by treatment with two combination of 50 μg of mAb 20B1 & 14G8 and 14G8 and 6D3 (p=0.0241). N=10 each group.

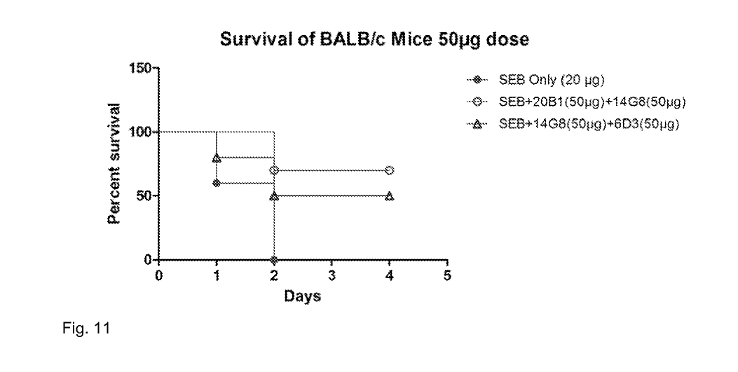

FIG. 12: CDR regions (in order, from top to bottom of each chain, CDR1, CDR2 and CDR3) of 20B1 IgG1 Vₕ and Vₗ sequences. Amino acid sequence of Vₕ is SEQ ID NO:18. Amino acid sequence of Vₗ is SEQ ID NO:19. Nucleotide sequence encoding Vₕ is SEQ ID NO:20. Nucleotide sequence encoding Vₗ is SEQ ID NO:21.

FIG. 13: CDR regions (in order, from top to bottom of each chain, CDR1, CDR2 and CDR3) of 6D3 IgG1 Vₕ and Vₗ sequences Amino acid sequence of Vₕ is SEQ ID NO:22. Amino acid sequence of Vₗ is SEQ ID NO:23. Nucleotide sequence encoding Vₕ is SEQ ID NO:24. Nucleotide sequence encoding Vₗ is SEQ ID NO:25.

FIG. 14: CDR regions (in order, from top to bottom of each chain, CDR1, CDR2 and CDR3) of 14G8 IgG1 Vₕ and Vₗ sequences. Amino acid sequence of Vₕ is SEQ ID NO:26. Amino acid sequence of Vₗ is SEQ ID NO:27. Nucleotide sequence encoding Vₕ is SEQ ID NO:28. Nucleotide sequence encoding Vₗ is SEQ ID NO:29.

FIG. 15: Survival of BALB/c mice from S. aureus infection (i.v.) Mice that underwent treatment with SEB-specific mAb 20B1 survived significantly longer compared to those mice treated with PBS treated mice (p=0.003).

FIG. 16: No difference in S. aureus CFU cultured between liver and spleen from treated or untreated mice at any tested time points.

FIG. 17: Mice infected i.v. with an SEB-producing MRSA strain and observed for 15 days. Significant survival differences in SEB immunized mice were documented compared to sham immunized mice (p=0.012).

FIG. 18: Histological examinations revealed wounds of mice infected SEB producing MRSA strain and treated with control mAb had high inflammation. Tissue Gram stains of these samples revealed large numbers of Gram positive cocci compare to SEB-specific mAb+SEB producing MRSA strain. Tissue sections from the wounds of mice infected with SEB-non-producing strains had no difference in inflammation and bacterial burden if treated with SEB-specific mAb or control mAb (data not shown).

### DETAILED DESCRIPTION OF THE INVENTION

Abbreviations used herein:

SE—Staphylococcal enterotoxin;
SEB—Staphylococcal enterotoxin B;
TSST-1—toxic shock syndrome toxin;
SEBILS—SEB-induced lethal shock;
Ab—antibody;
mAb—monoclonal antibody;
FcγR—Fc gamma receptor.

An isolated antibody, or an isolated antigen-binding fragment of an antibody, is provided which antibody or antigen-binding fragment binds to staphylococcal enterotoxin B (SEB) and which antibody or antigen-binding fragment comprises a heavy chain variable CDR3 comprising the sequence RIYYGNNGGVMDY (SEQ ID NO:30); ARTAGLLAPMDY (SEQ ID NO:31); ARDTMRKCYCELKLKPPAEHPGPA (SEQ ID NO:32) or VRDLYGDYVGRYAY (SEQ ID NO:48). In an embodiment, the SEB comprises SEQ ID NO:1. In an embodiment, the antibody or the antigen-binding fragment, comprises two heavy chain variable CDR3s each comprising the sequence RIYYGNNGGVMDY (SEQ ID NO:30); ARTAGLLAPMDY (SEQ ID NO:31); ARDTMRKCYCELKLKPPAEHPGPA (SEQ ID NO:32); or VRDLYGDYVGRYAY (SEQ ID NO:48).

In an embodiment, the antibody or the antigen-binding fragment, comprises a light chain variable CDR3 comprising the sequence LQYANYPWT (SEQ ID NO:33); QNDYTYPLT (SEQ ID NO:34); or QNGHSFPYT (SEQ ID NO:35). In an embodiment, the antibody or the antigen-binding fragment, comprises two light chain variable CDR3s each comprising the sequence LQYANYPWT (SEQ ID NO:33); QNDYTYPLT (SEQ ID NO:34); or QNGHSFPYT (SEQ ID NO:35).

In an embodiment, the antibody or the antigen-binding fragment, comprises a heavy chain variable CDR1 comprising the sequence GYIFTIAG (SEQ ID NO:36); GYTFTSHW (SEQ ID NO:37); GFTFSSYG (SEQ ID NO:38); or GFTFSAYG (SEQ ID NO:49).

In an embodiment, the antibody or the antigen-binding fragment, comprises a heavy chain variable CDR2 comprising the sequence INTHSGVP (SEQ ID NO:39); IDPSDSYI (SEQ ID NO:40); INSNGGST (SEQ ID NO:41); or ISGGGSV (SEQ ID NO:50).

In an embodiment, the antibody or the antigen-binding fragment, comprises two heavy chain variable CDR1s each comprising the sequence GYIFTIAG (SEQ ID NO:36); GYTFTSHW (SEQ ID NO:37); GFTFSSYG (SEQ ID NO:38); or GFTFSAYG (SEQ ID NO:49). In an embodiment, the antibody or the antigen-binding fragment, comprises two heavy chain variable CDR2s each comprising the sequence INTHSGVP (SEQ ID NO:39); IDPSDSYI (SEQ ID NO:40); INSNGGST (SEQ ID NO:41); or ISGGGSV (SEQ ID NO:50).

In an embodiment, the antibody or the antigen-binding fragment, comprises a light chain variable CDR1 comprising the sequence QEISDY (SEQ ID NO:42); QSLFNSGNQKNF (SEQ ID NO:43); or QSIGDY (SEQ ID NO:44). In an embodiment, the antibody or the antigen-binding fragment, comprises a light chain variable CDR2 comprising the sequence VAS (SEQ ID NO:45); WAS (SEQ ID NO:46); or YAS (SEQ ID NO:47).

In an embodiment, the antibody or the antigen-binding fragment, comprises two light chain variable CDR1s each comprising the sequence QEISDY (SEQ ID NO:42); QSLFNSGNQKNF (SEQ ID NO:43); or QSIGDY (SEQ ID NO:44). In an embodiment, the antibody or the antigen-binding fragment, comprises two light chain variable CDR2s each comprising the sequence VAS (SEQ ID NO:45); WAS (SEQ ID NO:46); or YAS (SEQ ID NO:47). In an embodiment, the antibody or the antigen-binding fragment, comprises a heavy chain variable CDR1 comprising the sequence GYIFTIAG (SEQ ID NO:36), a heavy chain variable CDR2 comprising the sequence INTHSGVP (SEQ ID NO:39), and a heavy chain variable CDR3 comprising the sequence RIYYGNNGGVMDY (SEQ ID NO:30). In an embodiment, the antibody or the antigen-binding fragment, comprises a light chain variable CDR1 comprising the sequence QEISDY (SEQ ID NO:42), a light chain variable CDR2 comprising the sequence VAS (SEQ ID NO:45), and a light chain variable CDR3 comprising the sequence LQYANYPWT (SEQ ID NO:33). In an embodiment, the antibody or the antigen-binding fragment, comprises two heavy chain variable CDR1s each comprising the sequence GYIFTIAG (SEQ ID NO:36), two heavy chain variable CDR2s each comprising the sequence INTHSGVP (SEQ ID NO:39), and two heavy chain variable CDR3s each comprising the sequence RIYYGNNGGVMDY (SEQ ID NO:30). In an embodiment, the antibody or the antigen-binding fragment, comprises two light chain variable CDR1s each comprising the sequence QEISDY (SEQ ID NO:42), two light chain variable CDR2s each comprising the sequence VAS (SEQ ID NO:45), and two light chain variable CDR3s each comprising the sequence LQYANYPWT (SEQ ID NO:33). In an embodiment, the antibody or the antigen-binding fragment, comprises a heavy chain variable CDR1 comprising the sequence GYTFTSHW (SEQ ID NO:37), a heavy chain variable CDR2 comprising the sequence IDPSDSYI (SEQ ID NO:40), and a heavy chain variable CDR3 comprising the sequence ARTAGLLAPMDY (SEQ ID NO:31). In an embodiment, the antibody or the antigen-binding fragment, comprises a light chain variable CDR1 comprising the sequence QSLFNSGNQKNF (SEQ ID NO:43), a light chain variable CDR2 comprising the sequence WAS (SEQ ID NO:46), and a light chain variable CDR3 comprising the sequence QNDYTYPLT (SEQ ID NO:34). In an embodiment, the antibody or the antigen-binding fragment, comprises two heavy chain variable CDR1s each comprising the sequence GYTFTSHW (SEQ ID NO:37), two heavy chain variable CDR2s each comprising the sequence IDPSDSYI (SEQ ID NO:40), and two heavy chain variable CDR3s each comprising the sequence ARTAGLLAPMDY (SEQ ID NO:31). In an embodiment, the antibody or the antigen-binding fragment, comprises two light chain variable CDR1s each comprising the sequence QSLFNSGNQKNF (SEQ ID NO:43), two light chain variable CDR2s each comprising the sequence WAS (SEQ ID NO:46), and two light chain variable CDR3s each comprising the sequence QNDYTYPLT (SEQ ID NO:34). In an embodiment, the antibody or the antigen-binding fragment, comprises a heavy chain variable CDR1 comprising the sequence GFTFSAYG (SEQ ID NO:49), a heavy chain variable CDR2 comprising the sequence ISGGGSV (SEQ ID NO:50), and a heavy chain variable CDR3 comprising the sequence VRDLYGDYVGRYAY (SEQ ID NO:48). In an embodiment, the antibody or the antigen-binding fragment, comprises a light chain variable CDR1 comprising the sequence QSIGDY (SEQ ID NO:44), a light chain variable CDR2 comprising the sequence YAS (SEQ ID NO:47), and a light chain variable CDR3 comprising the sequence QNGHSFPYT (SEQ ID NO:35). In an embodiment, the antibody or the antigen-binding fragment, comprises two heavy chain variable CDR1s each comprising the sequence GFTFSAYG (SEQ ID NO:49), two heavy chain variable CDR2s each comprising the sequence ISGGGSV (SEQ ID NO:50), and two heavy chain variable CDR3s each comprising the sequence VRDLYGDYVGRYAY (SEQ ID NO:48). In an embodiment, the antibody or the antigen-binding fragment, comprises two light chain variable CDR1s each comprising the sequence QSIGDY (SEQ ID NO:44), two light chain variable CDR2s each comprising the sequence YAS (SEQ ID NO:47), and two light chain variable CDR3s each comprising the sequence QNGHSFPYT (SEQ ID NO:35).

Also provided is a composition comprising any of the antibodies and/or antigen-binding fragments described herein. In an embodiment, the composition comprises two or more antibodies or antigen-binding fragments as described herein, wherein each of the antibodies or antigen-binding fragments comprise different sequences. In an embodiment, the composition is a pharmaceutical composition.

Also provided is an isolated antibody, or an isolated fragment of an antibody, which antibody or fragment (i) binds to staphylococcal enterotoxin B (SEB) comprising SEQ ID NO:1 and does not bind to the polypeptide set forth in SEQ ID NO:2, and (ii) recognizes residues 135, 137, 186, 235 and 236 of SEQ ID NO:1; residues 135, 137, 186, 188, 231, 233, 235 and 236 of SEQ ID NO:1; residues 135 and 186 of SEQ ID NO:1; or residues 135, 137, 186, 188, 235 and 236 of SEQ ID NO:1.

Also provided is an isolated antibody, or an isolated fragment of an antibody, which antibody or fragment binds to staphylococcal enterotoxin B (SEB) comprising SEQ ID NO:1 and which antibody or fragment

(i) exhibits reduced binding to a modified SEB compared to its binding to SEB comprising SEQ ID NO:1, wherein the modified SEB is modified relative to SEB comprising SEQ ID NO:1 by having any one or more of the residues numbered 135, 137, 186, 235 and 236 of SEQ ID NO:1 mutated to an alanine, but which does not exhibit reduced binding if the residue numbered 231 of SEQ ID NO:1 is mutated to an alanine; or
(ii) exhibits reduced binding to a modified SEB compared to its binding to SEB comprising SEQ ID NO:1, wherein the modified SEB is modified relative to SEB comprising SEQ ID NO:1 by having any one or more of the residues numbered 135 and 186 of SEQ ID NO:1 mutated to an alanine, but which does not exhibit reduced binding if the residue numbered 231 of SEQ ID NO:1 is mutated to an alanine;
(iii) exhibits reduced binding to a modified SEB compared to its binding to SEB comprising SEQ ID NO:1, wherein the modified SEB is modified relative to SEB comprising SEQ ID NO:1 by having any one or more of the residues numbered 135, 137, 186, 188, 235 and 236 of SEQ ID NO:1 mutated to an alanine, but which exhibits increased binding to a second modified SEB compared to binding to SEB comprising SEQ ID NO:1, wherein the second modified SEB is modified relative to SEQ ID NO:1 by having any one or more of the residues numbered 229, 233 or 231 of SEQ ID NO:1 mutated to an alanine; or
(iv) exhibits reduced binding to a modified SEB compared to its binding to SEB comprising SEQ ID NO:1, wherein the modified SEB is modified relative to SEB comprising SEQ ID NO:1 by having any one or more of the residues numbered 135, 137, 186, 235 and 236 of SEQ ID NO:1 mutated to an alanine, but which does not exhibit reduced binding if one or more of the residues numbered 188, 231 or 233 of SEQ ID NO:1 is mutated to an alanine.

In an embodiment, in (i) the antibody or the fragment also does not exhibit reduced binding if the residue numbered 188 of SEQ ID NO:1 is mutated to an alanine. In an embodiment, in (i) the antibody or the fragment also does not exhibit reduced binding if the residue numbered 233 of SEQ ID NO:1 is mutated to an alanine. In an embodiment, in (ii) the antibody or the fragment also does not exhibit reduced binding if the residue numbered 188 of SEQ ID NO:1 is mutated to an alanine.

In an embodiment, the antibody or fragment does not bind to a modified staphylococcal enterotoxin B, which modified staphylococcal enterotoxin B is modified relative to unmodified staphylococcal enterotoxin B comprising SEQ ID NO:1 by not comprising the C-terminal eleven amino acid residues of SEQ ID NO:1.

Also provided is an isolated antibody or the antigen-binding fragment of an antibody of any of the described antibodies, wherein the antibody is a human antibody, a humanized antibody or a chimeric antibody. In an embodiment, the antibody is a monoclonal antibody. In an embodiment, the antibody is a human antibody. In an embodiment, the fragment is of a human antibody, of a humanized antibody or of a chimeric antibody. In an embodiment, the fragment is of a monoclonal antibody. In an embodiment, the fragment is of a human antibody. In an embodiment, the fragment comprises an Fab, an Fab′, an F(ab′)₂, an F_{d}, an Fᵥ, a complementarity determining region (CDR), or a single-chain antibody (scFv). In an embodiment, the antigen is SEB.

In an embodiment, a heavy chain of the antibody or fragment is encoded by a germline gene of the V_{H} AJ972403, V_{H} X03399 family or X00160 family.

In an embodiment, the isolated antibody or the isolated fragment of an antibody does not bind staphylococcal enterotoxin A (SEA). In an embodiment, the isolated antibody or the isolated fragment of an antibody does not bind toxic shock syndrome toxin-1 (TSST-1).

In an embodiment, the isolated antibody or the isolated fragment of an antibody exhibits reduced binding to a modified SEB compared to binding to SEB comprising SEQ ID NO:1, wherein the modified SEB is modified relative to SEB comprises SEQ ID NO:1 by having any one or more of the residues numbered 135, 137, 186, 235 and 236 of SEQ ID NO:1 mutated to an alanine, but which does not exhibit reduced binding if the residue numbered 231 of SEQ ID NO:1 is mutated to an alanine.

In an embodiment, the isolated antibody or the isolated fragment of an antibody exhibits reduced binding to a modified SEB compared to binding to SEB comprising SEQ ID NO:1, wherein the modified SEB is modified relative to SEB comprising SEQ ID NO:1 by having any one or more of the residues numbered 135 and 186 of SEQ ID NO:1 mutated to an alanine, but which does not exhibit reduced binding if the residue numbered 231 of SEQ ID NO:1 is mutated to an alanine.

In an embodiment, the isolated antibody or the isolated fragment of an antibody exhibits reduced binding to a modified SEB compared to binding to SEB comprising SEQ ID NO:1, wherein the modified SEB is modified relative to SEB comprising SEQ ID NO:1 by having any one or more of the residues numbered 135, 137, 186, 188, 235 and 236 of SEQ ID NO:1 mutated to an alanine, but which exhibits increased binding to a second modified SEB compared to binding to SEB comprising SEQ ID NO:1, wherein the second modified SEB is modified relative to SEB comprising SEQ ID NO:1 by having any one or more of the residues numbered 229, 233 or 231 of SEQ ID NO:1 mutated to an alanine.

In an embodiment, the isolated antibody or the isolated fragment of an antibody exhibits reduced binding to a modified SEB compared to binding to SEB comprising SEQ ID NO:1, wherein the modified SEB is modified relative to SEB comprising SEQ ID NO:1 by having any one or more of the residues numbered 135, 137, 186, 235 and 236 of SEQ ID NO:1 mutated to an alanine, but which does not exhibit reduced binding if one or more of the residues numbered 188, 231 or 233 of SEQ ID NO:1 is mutated to an alanine.

A composition is provided comprising any of the described isolated antibodies or the described isolated fragments of an antibody. A pharmaceutical composition is provided comprising any of the described isolated antibodies or the described isolated fragments of an antibody.

Also provided is a method of treating a disease associated with a staphylococcus infection in a subject having the disease, or preventing a disease associated with a staphylococcus infection in a subject at risk of the disease, comprising administering to the subject an amount of an antibody directed against SEB or antigen-binding fragment thereof as described herein, or an amount of a antibody or antigen-binding fragment thereof directed to a conformational epitope of staphylococcal enterotoxin B (SEB), effective to treat the disease. In an embodiment, the antibody is a monoclonal antibody. In an embodiment, the amount of an antibody directed against SEB or antigen-binding fragment thereof as described herein is administered.

Also provided is a method of treating a disease associated with a staphylococcus infection in a subject having the disease, or preventing a disease associated with a staphylococcus infection in a subject at risk of the disease, comprising administering to the subject a monoclonal antibody or antigen-binding fragment thereof directed to a conformational epitope of staphylococcal enterotoxin B (SEB) effective to treat the disease.

In an embodiment, the antibody is a monoclonal antibody. In an embodiment, the amount of an antibody directed against SEB or antigen-binding fragment thereof as described herein is administered. In an embodiment, the SEB comprises SEQ ID NO:1.

In an embodiment, the monoclonal antibody or antigen-binding fragment thereof does not bind to a modified staphylococcal enterotoxin B which is modified relative to SEB comprising SEQ ID NO:1 by not comprising the C-terminal ten amino acid residues of the SEQ ID NO:1. In an embodiment, at least two different antibodies or antigen-binding fragments thereof directed to a conformational epitope of SEB are administered and their amounts combined are effective to treat the disease.

In an embodiment, the disease is sepsis, SEB-mediated shock, a staphylococcus aureus infection, staphylococcus aureus bacteremia, or staphylococcus aureus-associated atopic dermatitis. In an embodiment, the disease is staphylococcus aureus infection. In an embodiment, the disease is staphylococcus aureus skin infection. In an embodiment, the staphylococcus aureus is methicillin-resistant staphylococcus aureus. In an embodiment, the staphylococcus aureus is methicillin-sensitive staphylococcus aureus.

In an embodiment, one antibody is neutralizing and the other antibody is not neutralizing. In an embodiment, both antibodies are neutralizing. In an embodiment, neither antibody alone is neutralizing.

In an embodiment, at least one administered monoclonal antibody or antigen-binding fragment thereof recognizes residues 135, 137, 186, 235 and 236 of SEQ ID NO:1; residues 135, 137, 186, 188, 231, 233, 235 and 236 of SEQ ID NO:1; residues 135 and 186 of SEQ ID NO:1; or residues 135, 137, 186, 188, 235 and 236 of SEQ ID NO:1.

In an embodiment, at least one administered monoclonal antibody or antigen-binding fragment thereof recognizes residues 135, 137, 186, 235 and 236 of SEQ ID NO:1; and another of the administered monoclonal antibodies or antigen-binding fragments thereof recognizes residues 135, 137, 186, 188, 231, 233, 235 and 236 of SEQ ID NO:1; residues 135 and 186 of SEQ ID NO:1; or residues 135, 137, 186, 188, 235 and 236 of SEQ ID NO:1.

In an embodiment, at least one administered monoclonal antibody or antigen-binding fragment thereof

(i) exhibits reduced binding to a modified SEB compared to its binding to SEB comprising SEQ ID NO:1, wherein the modified SEB is modified relative to SEB comprising SEQ ID NO:1 by having any one or more of the residues numbered 135, 137, 186, 235 and 236 of SEQ ID NO:1 mutated to an alanine, but which does not exhibit reduced binding if the residue numbered 231 of SEQ ID NO:1 is mutated to an alanine; or
(ii) exhibits reduced binding to a modified SEB compared to its binding to SEB comprising SEQ ID NO:1, wherein the modified SEB is modified relative to SEB comprising SEQ ID NO:1 by having any one or more of the residues numbered 135 and 186 of SEQ ID NO:1 mutated to an alanine, but which does not exhibit reduced binding if the residue numbered 231 of SEQ ID NO:1 is mutated to an alanine.

In an embodiment, in (i) the antibody or the fragment does not exhibit reduced binding if the residue numbered 188 of SEQ ID NO:1 is mutated to an alanine. In an embodiment, in (i) the antibody or the fragment does not exhibit reduced binding if the residue numbered 233 of SEQ ID NO:1 is mutated to an alanine. In an embodiment, in (ii) the antibody or the fragment does not exhibit reduced binding if the residue numbered 188 of SEQ ID NO:1 is mutated to an alanine.

A method is provided of treating a disease associated with a staphylococcus infection in a subject having the disease, or of preventing a disease associated with a staphylococcus infection in a subject at risk thereof, comprising administering to the subject an amount of at least two different antibodies or antigen-binding fragments thereof each directed against SEB as described herein, or an amount of at least two different antibodies or antigen-binding fragments thereof each recognizing a conformational epitope of SEB, effective to treat the disease. In an embodiment, the SEB comprises SEQ ID NO:1. In an embodiment, the antibodies are monoclonal antibodies. In an embodiment, the antigen-binding fragments are fragments of monoclonal antibodies. In an embodiment, the antibodies or fragments each recognize a conformational epitope of SEB. In an embodiment, the monoclonal antibodies or antigen-binding fragments each do not bind to a modified staphylococcal enterotoxin B which is modified relative to SEB comprising SEQ ID NO:1 by not comprising the C-terminal ten amino acid residues of the SEQ ID NO:1.

In an embodiment of the methods, the disease is sepsis, SEB-mediated shock, a staphylococcus aureus infection, bacteremia, or staphylococcus aureus-associated atopic dermatitis. In an embodiment, the disease is a staphylococcus aureus infection. In an embodiment, the disease is a staphylococcus aureus skin infection. In an embodiment, the disease is a staphylococcus aureus bacteremia. In an embodiment, the staphylococcus aureus is methicillin-resistant staphylococcus aureus. In an embodiment, the staphylococcus aureus is methicillin-sensitive staphylococcus aureus. In an embodiment, one antibody is neutralizing and the other antibody is not neutralizing. In an embodiment, both antibodies are neutralizing. In an embodiment, neither antibody alone is neutralizing. In an embodiment, at least one administered monoclonal antibody or antigen-binding fragment thereof is an antibody or antigen-binding fragment as described herein. In an embodiment, the antibody or antigen-binding fragment thereof is, or antibodies or antigen-binding fragments thereof are, administered prophylactically. In an embodiment, the antibody or antigen-binding fragment thereof is, or antibodies or antigen-binding fragments thereof are, administered after the disease has manifested.

In an embodiment, the subject is administered an antibody or antibodies and the antibody or antibodies are, chimeric monoclonal antibodies, humanized monoclonal antibodies or human monoclonal antibodies. In an embodiment, the subject is administered an antigen-binding fragment of an antibody or antigen-binding fragments of antibodies and the antigen-binding fragment or antigen-binding fragments are fragments of chimeric monoclonal antibodies, humanized monoclonal antibodies or human monoclonal antibodies.

Also provided is a method for identifying a candidate agent as an agent for treating a disease associated with a staphylococcus infection comprising contacting staphylococcal enterotoxin B (SEB) comprising SEQ ID NO:1 with the candidate agent and determining if the candidate agent binds to, or competes with an antibody binding to, residues 135, 137, 186, 235 and 236 of SEQ ID NO:1; residues 135, 137, 186, 188, 231, 233, 235 and 236 of SEQ ID NO:1; residues 135 and 186 of SEQ ID NO:1; or residues 135, 137, 186, 188, 235 and 236 of SEQ ID NO:1,

wherein if the candidate agent binds to, or competes with the antibody binding to, residues 135, 137, 186, 235 and 236 of SEQ ID NO:1; residues 135, 137, 186, 188, 231, 233, 235 and 236 of SEQ ID NO:1; residues 135 and 186 of SEQ ID NO:1; or residues 135, 137, 186, 188, 235 and 236 of SEQ ID NO:1 then the candidate agent is identified as an agent for treating a disease associated with a staphylococcus infection and wherein if the candidate agent does not bind to, or does not compete with the antibody binding to, residues 135, 137, 186, 235 and 236 of SEQ ID NO:1; residues 135, 137, 186, 188, 231, 233, 235 and 236 of SEQ ID NO:1; residues 135 and 186 of SEQ ID NO:1; or residues 135, 137, 186, 188, 235 and 236 of SEQ ID NO:1, then the candidate agent is not identified as an agent for treating a disease associated with a staphylococcus infection.

In an embodiment of the method, the agent is an antibody, a fragment of an antibody or a peptide. In an embodiment, the agent is a small molecule.

An isolated antibody is provided which inhibits SEB-induced human T-cell proliferation and SEB-induced human T-cell IL-2 and IFN-γ production when bound to a human T-cell. In an embodiment, the antibody is a human antibody, a humanized antibody or a chimeric antibody. In an embodiment, the antibody is a monoclonal antibody. In an embodiment, the antibody is a human antibody. In an embodiment, the antibody is a humanized antibody.

Also provided is an isolated antibody, or the isolated antigen-binding fragment of an antibody, which antibody or antigen-binding fragment binds to staphylococcal enterotoxin B (SEB) and which antibody or antigen-binding fragment comprises an amino acid sequence comprising three CDRs, each one of which has at least 90% identity to a different heavy chain variable CDR selected from CDR1, CDR2 and CDR3 as set forth in SEQ ID NOS:36, 39, and 30, or from CDR1, CDR2 and CDR3 as set forth in SEQ ID NOS:37, 40, and 31, or from CDR1, CDR2 and CDR3 as set forth in SEQ ID NOS:49, 50, and 48. In an embodiment, one, two, or three of the CDRs, each have at least one of 91%, 92%, 93%, 94%, 95%, 96%, 97%, 98% or 99% identity to a different heavy chain variable CDR selected from CDR1, CDR2 and CDR3 as set forth in SEQ ID NOS:36, 39, and 30, or from CDR1, CDR2 and CDR3 as set forth in SEQ ID NOS:37, 40, and 31, or from CDR1, CDR2 and CDR3 as set forth in SEQ ID NOS:49, 50, and 48. In an embodiment, the antibody or antigen-binding fragment comprises an amino acid sequence comprising three CDRs, each one of which has at least 90% identity to a different light chain variable CDR selected from CDR1, CDR2 and CDR3 as set forth in SEQ ID NOS:42, 45 or 33, or from CDR1, CDR2 and CDR3 as set forth in SEQ ID NOS:43, 46 or 34, or from CDR1, CDR2 and CDR3 as set forth in SEQ ID NOS:44, 47 or 35. In an embodiment, one, two, or three of the CDRs, each have at least one of 91%, 92%, 93%, 94%, 95%, 96%, 97%, 98% or 99% identity to a different light chain variable CDR selected from CDR1, CDR2 and CDR3 as set forth in SEQ ID NOS:42, 45 or 33, or from CDR1, CDR2 and CDR3 as set forth in SEQ ID NOS:43, 46 or 34, or from CDR1, CDR2 and CDR3 as set forth in SEQ ID NOS:44, 47 or 35. In an embodiment, the isolated antibody, or the isolated antigen-binding fragment of an antibody, comprises two of the heavy chain CDR1, CDR2 and CDR3 and two of the light chain CDR1, CDR2 and CDR3. In an embodiment, the SEB comprises SEQ ID NO:1. In an embodiment, the antibody or antigen-binding fragment of the antibody binds SEB with an affinity of <400 pM.

The antigen, in regard to the antigen-binding fragment, is SEB.

Also provided is an isolated antibody, or the isolated antigen-binding fragment of an antibody, which antibody or antigen-binding fragment binds to staphylococcal enterotoxin B (SEB) comprising SEQ ID NO:1 and which antibody or antigen-binding fragment comprises an amino acid sequence comprising three CDRs, each one of which is at least 90%, or at least 95%, homologous to a different heavy chain variable CDR selected from CDR1, CDR2 and CDR3 as set forth in SEQ ID NO:18, 22, or 26.

In an embodiment, the antibody or antigen-binding fragment comprises an amino acid sequence comprising three CDRs, each one of which is at least 90%, or at least 95%, homologous to a different light chain variable CDR selected from CDR1, CDR2 and CDR3 as set forth in SEQ ID NO:19, 23 or 27.

In an embodiment, the antibody or antigen-binding fragment comprises two heavy chains and two light chains, each heavy chain comprising three CDRs, each one of which is at least 90%, or at least 95%, homologous to a different heavy chain variable CDR selected from CDR1, CDR2 and CDR3 as set forth in SEQ ID NO:18, 22, or 26, and each light chain comprising three CDRs, each one of which is at least 90%, or at least 95%, homologous to a different light chain variable CDR selected from CDR1, CDR2 and CDR3 as set forth in SEQ ID NO:19, 23 or 27.

In an embodiment, the antibody or the antigen-binding fragment CDRs are 100% homolgous to their respective CDRs set forth in SEQ ID NO:18, 22, 26, 19, 23 and 27.

Also provided is an isolated antibody, or an isolated antigen-binding fragment of an antibody, which antibody or antigen-binding fragment binds to staphylococcal enterotoxin B (SEB) and which comprises (a) two heavy chains each comprising the sequence set forth in SEQ ID NO:8, 22, or 26, and (b) two light chains each comprising the sequence set forth in SEQ ID NO:19, 23 and 27.

In an embodiment, the antibody or fragment comprises (a) two heavy chains each comprising the sequence set forth in SEQ ID NO:18, and (b) two light chains each comprising the sequence set forth in SEQ ID NO:19. In an embodiment, the antibody or fragment comprises (a) two heavy chains each comprising the sequence set forth in SEQ ID NO:22, and (b) two light chains each comprising the sequence set forth in SEQ ID NO:23. In an embodiment, the antibody or fragment comprises (a) two heavy chains each comprising the sequence set forth in SEQ ID NO:26, and (b) two light chains each comprising the sequence set forth in SEQ ID NO:27. In an embodiment, the antibody or fragment, is a human antibody, a humanized antibody or a chimeric antibody or fragment thereof. In an embodiment, the antibody is a monoclonal antibody or the fragment is a fragment of a monoclonal antibody. In an embodiment, the antibody is a human antibody or the fragment is a fragment of a human antibody. In an embodiment, the antibody is a humanized antibody or the fragment is a fragment of a humanized antibody.

A composition is provided comprising any of the antibody or antigen-binding fragments described herein. In an embodiment, the composition is a pharmaceutical composition and comprises a pharmaceutically acceptable carrier.

Also provided is an antibody or antigen binding fragment of an antibody as described herein, for treating a staphylococcus aureus infection, staphylococcus aureus bacteremia, staphylococcus aureus-associated sepsis, SEB-mediated shock, or staphylococcus aureus-associated atopic dermatitis in a subject. In an embodiment, the disease is a staphylococcus aureus skin infection.

In an embodiment of the antibodies, fragments, methods and compositions described herein, the antibody, or the antigen-binding fragment, has an affinity for SEB of less than 400 pM. In an embodiment, the antibody, or the antigen-binding fragment, has an affinity for SEB of less than 375 pM. In an embodiment, the antibody, or the antigen-binding fragment, has an affinity for SEB of less than 360 pM. In an embodiment, the antibody, or the antigen-binding fragment, has an affinity for SEB of less than 325 pM. In an embodiment, the antibody, or the antigen-binding fragment, has an affinity for SEB 310 pM or less.

In an embodiment of the antibodies, fragments, methods and compositions described herein, the SEB has the sequence set forth in SEQ ID NO:1.

Also provided is an antibody, or antigen-binding fragment of an antibody, as described herein, for treating a disease associated with a staphylococcus infection in a subject, wherein the antibody or antibody fragment is administered concurrently, separately or sequentially with a second antibody or second antibody fragment directed against SEB, wherein the second antibody or second antibody fragment is of a different sequence than the antibody, or antigen-binding fragment of an antibody. In an embodiment, the second antibody or second antibody fragment is also as described herein. Also provided is a first antibody, or antigen-binding fragment thereof, and a second antibody, or antigen-binding fragment thereof, wherein the first and second antibody or fragments thereof are as described herein, and wherein the first and second antibody, or fragments thereof, have different sequences, as a combined preparation for treating a disease associated with a staphylococcus infection in a subject. Also provided is a first antibody, or antigen-binding fragment thereof, for use with a second antibody, or antigen-binding fragment thereof, wherein the first and second antibody or fragments thereof are as described herein and wherein the first and second antibody, or fragments thereof, have different sequences, for treating a disease associated with a staphylococcus infection in a subject. In an embodiment, the disease is a staphylococcus aureus infection, staphylococcus aureus bacteremia, staphylococcus aureus-associated sepsis, SEB-mediated shock, or staphylococcus aureus-associated atopic dermatitis. In an embodiment, the disease is a staphylococcus aureus skin infection.

In an embodiment of the antibodies, fragments, methods and compositions described herein, the fragment comprises an Fab, an Fab′, an F(ab′)₂, an F_{d}, an Fᵥ, a complementarity determining region (CDR), or a single-chain antibody (scFv). In an embodiment, the fragment comprises a CDR3 of a Vₕ chain. In an embodiment the fragment also comprises one of, more than one of, or all of CDR1, CDR2 of Vₕ and CDR1, CDR2 and CDR3 of a Vₗ. In an embodiment, a heavy chain of the antibody or fragement is encoded by a germline gene of the V_{H}7183 family.

As used herein, “neutralizing” means toxin-neutralizing, specifically, the toxin SEB.

In an embodiment of the methods, the antibody or antigen-binding fragment thereof is, or antibodies or antigen-binding fragments thereof are, administered prophylactically. In an embodiment, the antibody or antigen-binding fragment thereof is, or antibodies or antigen-binding fragments thereof are, administered after the disease has manifested. In an embodiment, the subject is administered an antibody or antibodies and the antibody or antibodies are, chimeric monoclonal antibodies, humanized monoclonal antibodies or human monoclonal antibodies.

In an embodiment, the subject is administered an antigen-binding fragment of an antibody or antigen-binding fragments of antibodies and the antigen-binding fragment or antigen-binding fragments are fragments of chimeric monoclonal antibodies, humanized monoclonal antibodies or human monoclonal antibodies.

In an embodiment of the methods, at least one antibody and one antigen-binding fragment of an antibody are administered to the subject.

In an embodiment of the methods, the antibody, antibodies, antibody fragment or antibody fragments are administered as an adjuvant therapy to a primary therapy for the disease or condition.

A method is provided for identifying a candidate agent as an agent for treating a disease associated with a staphylococcus infection comprising contacting staphylococcal enterotoxin B (SEB) comprising SEQ ID NO:1 with the candidate agent and determining if the candidate agent binds to, or competes with an antibody binding to, residues 135, 137, 186, 235 and 236 of SEQ ID NO:1; residues 135, 137, 186, 188, 231, 233, 235 and 236 of SEQ ID NO:1; residues 135 and 186 of SEQ ID NO:1; or residues 135, 137, 186, 188, 235 and 236 of SEQ ID NO:1, wherein if the candidate agent binds to, or competes with the antibody binding to, residues 135, 137, 186, 235 and 236 of SEQ ID NO:1; residues 135, 137, 186, 188, 231, 233, 235 and 236 of SEQ ID NO:1; residues 135 and 186 of SEQ ID NO:1; or residues 135, 137, 186, 188, 235 and 236 of SEQ ID NO:1 then the candidate agent is an agent for treating a disease associated with a staphylococcus infection and wherein if the candidate agent does not bind to, or does not compete with the antibody binding to, residues 135, 137, 186, 235 and 236 of SEQ ID NO:1; residues 135, 137, 186, 188, 231, 233, 235 and 236 of SEQ ID NO:1; residues 135 and 186 of SEQ ID NO:1; or residues 135, 137, 186, 188, 235 and 236 of SEQ ID NO:1, then the candidate agent is not identified as an agent for treating a disease associated with a staphylococcus infection.

In an embodiment, the agent is an antibody, a fragment of an antibody or a peptide. In an embodiment, the agent is a small molecule.

Also provided is an isolated antibody which inhibits SEB-induced human T-cell proliferation and SEB-induced human T-cell IL-2 and IFN-γ production when bound to a human T-cell. In an embodiment, the antibody is a human antibody, a humanized antibody or a chimeric antibody. In an embodiment, the antibody is a monoclonal antibody. In an embodiment, the antibody is a human antibody. In an embodiment, the antibody is a humanized antibody.

Also provided is an isolated antibody, or an isolated antigen-binding fragment of an antibody, which antibody or antigen-binding fragment binds to staphylococcal enterotoxin B (SEB) comprising SEQ ID NO:1 and which antibody or antigen-binding fragment comprises a heavy chain variable CDR3 comprising RIYYGNNGGVMDY (SEQ ID NO:30); ARTAGLLAPMDY (SEQ ID NO:31); or ARDTMRKCYCELKLKPPAEHPGPA (SEQ ID NO:32).

In an embodiment, the antibody or the antigen-binding fragment comprises two heavy chain variable CDR3 each comprising RIYYGNNGGVMDY (SEQ ID NO:30); ARTAGLLAPMDY (SEQ ID NO:31); or ARDTMRKCYCELKLKPPAEHPGPA (SEQ ID NO:32). In an embodiment, the antibody or the antigen-binding fragment comprises a light chain variable CDR3 comprising LQYANYPWT (SEQ ID NO:33); QNDYTYPLT (SEQ ID NO:34); or QNGHSFPYT (SEQ ID NO:35).

In an embodiment, the antibody or the antigen-binding fragment two light chain variable CDR3 each comprising LQYANYPWT (SEQ ID NO:33); QNDYTYPLT (SEQ ID NO:34); or QNGHSFPYT (SEQ ID NO:35).

In an embodiment, the antibody or the antigen-binding fragment comprises a heavy chain variable CDR1 comprising GYIFTIAG (SEQ ID NO:36); GYTFTSHW (SEQ ID NO:37); or GFTFSSYG (SEQ ID NO:38).

In an embodiment, the antibody or the antigen-binding fragment comprises a heavy chain variable CDR2 comprising INTHSGVP (SEQ ID NO:39); IDPSDSYI (SEQ ID NO:40); or INSNGGST (SEQ ID NO:41).

In an embodiment, the antibody or the antigen-binding fragment comprises two heavy chain variable CDR1 each comprising GYIFTIAG (SEQ ID NO:36); GYTFTSHW (SEQ ID NO:37); or GFTFSSYG (SEQ ID NO:38).

In an embodiment, the antibody or the antigen-binding fragment comprises two heavy chain variable CDR2 each comprising INTHSGVP (SEQ ID NO:39); IDPSDSYI (SEQ ID NO:40); or INSNGGST (SEQ ID NO:41).

In an embodiment, the antibody or the antigen-binding fragment comprises a light chain variable CDR1 comprising QEISDY (SEQ ID NO:42); QSLFNSGNQKNF (SEQ ID NO:43); or QSIGDY (SEQ ID NO:44).

In an embodiment, the antibody or the antigen-binding fragment comprises a light chain variable CDR2 comprising VAS (SEQ ID NO:45); WAS (SEQ ID NO:46); or YAS (SEQ ID NO:47).

In an embodiment, the antibody or the antigen-binding fragment comprises two light chain variable CDR1 each comprising QEISDY (SEQ ID NO:42); QSLFNSGNQKNF (SEQ ID NO:43); or QSIGDY (SEQ ID NO:44).

In an embodiment, the antibody or the antigen-binding fragment comprises two light chain variable CDR2 each comprising VAS (SEQ ID NO:45); WAS (SEQ ID NO:46); or YAS (SEQ ID NO:47).

Also provides is an isolated antibody, or the isolated antigen-binding fragment of an antibody, which antibody or antigen-binding fragment binds to staphylococcal enterotoxin B (SEB) comprising SEQ ID NO:1 and which antibody or antigen-binding fragment comprises an amino acid sequence comprising three CDRs, each one of which is at least 90%, or at least 95%, homologous to a different heavy chain variable CDR selected from CDR1, CDR2 and CDR3 as set forth in SEQ ID NO:18, 22, or 26.

In an embodiment, the antibody or the antigen-binding fragment comprises an amino acid sequence comprising three CDRs, each one of which is at least 90%, or at least 95%, homologous to a different light chain variable CDR selected from CDR1, CDR2 and CDR3 as set forth in SEQ ID NO:19, 23 or 27.

In an embodiment, the antibody or the antigen-binding fragment comprises two heavy chains and two light chains, each heavy chain comprising three CDRs, each one of which is at least 90%, or at least 95%, homologous to a different heavy chain variable CDR selected from CDR1, CDR2 and CDR3 as set forth in SEQ ID NO:18, 22, or 26, and each light chain comprising three CDRs, each one of which is at least 90%, or at least 95%, homologous to a different light chain variable CDR selected from CDR1, CDR2 and CDR3 as set forth in SEQ ID NO:19, 23 or 27.

In an embodiment, the CDRs are 100% homolgous to their respective CDRs set forth in SEQ ID NO:18, 22, 26, 19, 23 and 27.

Also provided is an isolated antibody, or an isolated antigen-binding fragment of an antibody, which antibody or antigen-binding fragment binds to staphylococcal enterotoxin B (SEB) and which comprises (a) two heavy chains each comprising the sequence set forth in SEQ ID NO:8, 22, or 26, and (b) two light chains each comprising the sequence set forth in SEQ ID NO:19, 23 and 27.

In an embodiment, the antibody or the antigen-binding fragment comprises (a) two heavy chains each comprising the sequence set forth in SEQ ID NO:18, and (b) two light chains each comprising the sequence set forth in SEQ ID NO:19. In an embodiment, the antibody or the antigen-binding fragment comprises (a) two heavy chains each comprising the sequence set forth in SEQ ID NO:22, and (b) two light chains each comprising the sequence set forth in SEQ ID NO:23. In an embodiment, the antibody or the antigen-binding fragment comprises (a) two heavy chains each comprising the sequence set forth in SEQ ID NO:26, and (b) two light chains each comprising the sequence set forth in SEQ ID NO:27.

In an embodiment of the isolated antibody or the isolated antigen-binding fragment, the antibody is a human antibody, a humanized antibody or a chimeric antibody. In an embodiment of the antibody or the antigen-binding fragment, the antibody is a monoclonal antibody. In an embodiment of the antibody or the antigen-binding fragment, the antibody is a human antibody. In an embodiment of the antibody or the antigen-binding fragment, the antibody is a humanized antibody.

Also provided is a composition comprising any of the antibody or antigen-binding fragment of an antibody described herein. In an embodiment, the composition is a pharmaceutical composition and comprises a pharmaceutically acceptable carrier.

Also provided are methods of treating a disease associated with a staphylococcus infection in a subject having the disease or at risk of the disease comprising administering to the subject an amount of at least two different monoclonal antibodies as described herein or antigen-binding fragments thereof as described herein, wherein each monoclonal antibody is directed to staphylococcal enterotoxin B (SEB), effective to treat the disease. In embodiments, the disease is sepsis, SEB-mediated shock, or a staphylococcus aureus infection, bacteremia or staphylococcus aureus-associated atopic dermatitis. In an embodiment the staphylococcus aureus is methicillin-resistant staphylococcus aureus.

As used herein “sepsis” is the medically recognized condition characterized by a systemic inflammatory response to for example a pathogenic bacteria such as a staphylococcal pathogen.

As used herein, diseases “associated with staphylococcal infection” include boils, styes, furuncles, pneumonia, mastitis, phlebitis, meningitis, urinary tract infections, osteomyelitis, endocarditis, septicemia, and S. aureus nosocomial infection of surgical wounds and infections associated with indwelling medical devices. S. aureus can also cause food poisoning and toxic shock syndrome.

In an embodiment of the methods of treatment, the methods further comprise administering to the subject an antibiotic, optionally in combination with the antibody or fragments. In a preferred embodiment the antibiotic is an anti-staphylococcal antibiotic. In an embodiment, the antibiotic is effective against staphylococcus aureus.

As used herein, the term “antibody” refers to an intact antibody, i.e. with complete Fc and Fv regions. “Fragment” refers to any portion of an antibody, or portions of an antibody linked together, such as a single-chain Fv (scFv), which is less than the whole antibody but which is an antigen-binding portion and which competes with the intact antibody of which it is a fragment for specific binding. As such a fragment can be prepared, for example, by cleaving an intact antibody or by recombinant means. See generally, Fundamental Immunology, Ch. 7 (Paul, W., ed., 2nd ed. Raven Press, N.Y. (1989), hereby incorporated by reference in its entirety). Antigen-binding fragments may be produced by recombinant DNA techniques or by enzymatic or chemical cleavage of intact antibodies or by molecular biology techniques. In some embodiments, a fragment is an Fab, Fab′, F(ab′)₂, F_{d}, Fᵥ, complementarity determining region (CDR) fragment, single-chain antibody (scFv), (a variable domain light chain (V_{L}) and a variable domain heavy chain (V_{H}) linked via a peptide linker. In an embodiment the linker of the scFv is 10-25 amino acids in length. In an embodiment the peptide linker comprises glycine, serine and/or threonine residues. For example, see Bird et al., Science, 242: 423-426 (1988) and Huston et al., Proc. Natl. Acad. Sci. USA, 85:5879-5883 (1988) each of which are hereby incorporated by reference in their entirety), or a polypeptide that contains at least a portion of an antibody that is sufficient to confer SEB-specific antigen binding on the polypeptide, including a diabody. From N-terminus to C-terminus, both the mature light and heavy chain variable domains comprise the regions FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md. (1987 and 1991)), Chothia & Lesk, J. Mol. Biol. 196:901-917 (1987), or Chothia et al., Nature 342:878-883 (1989), each of which are hereby incorporated by reference in their entirety). As used herein, the term “polypeptide” encompasses native or artificial proteins, protein fragments and polypeptide analogs of a protein sequence. A polypeptide may be monomeric or polymeric. As used herein, an F_{d} fragment means an antibody fragment that consists of the V_{H} and CH1 domains; an Fᵥ fragment consists of the Vₗ and V_{H} domains of a single arm of an antibody; and a dAb fragment (Ward et al., Nature 341:544-546 (1989) hereby incorporated by reference in its entirety) consists of a V_{H} domain.

In some embodiments, fragments are at least 5, 6, 8 or 10 amino acids long. In other embodiments, the fragments are at least 14, at least 20, at least 50, or at least 70, 80, 90, 100, 150 or 200 amino acids long.

The term “monoclonal antibody” is not intended to be limited as regards to the source of the antibody or the manner in which it is made (e.g., by hybridoma, phage selection, recombinant expression, transgenic animals, etc.). The term “monoclonal antibody” as used herein refers to an antibody member of a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins. Thus an identified monoclonal antibody can be produced by non-hybridoma techniques, e.g. by appropriate recombinant means once the sequence thereof is identified.

As used herein, the terms “isolated antibody” refers to an antibody that by virtue of its origin or source of derivation has one to four of the following: (1) is not associated with naturally associated components that accompany it in its native state, (2) is free of other proteins from the same species, (3) is expressed by a cell from a different species, or (4) does not occur in nature.

In an embodiment the composition or pharmaceutical composition comprising one or more of the antibodies or fragments described herein is substantially pure with regard to the antibody or fragment. A composition or pharmaceutical composition comprising one or more of the antibodies or fragments described herein is “substantially pure” with regard to the antibody or fragment when at least about 60 to 75% of a sample of the composition or pharmaceutical composition exhibits a single species of the antibody or fragment. A substantially pure composition or pharmaceutical composition comprising one or more of the antibodies or fragments described herein can comprise, in the portion thereof which is the antibody or fragment, 60%, 70%, 80% or 90% of the antibody or fragment of the single species, more usually about 95%, and preferably over 99%. Antibody purity or homogeneity may tested by a number of means well known in the art, such as polyacrylamide gel electrophoresis or HPLC.

As used herein, a “human antibody” unless otherwise indicated is one whose sequences correspond to (i.e. are identical in sequence to) an antibody that could be produced by a human and/or has been made using any of the techniques for making human antibodies as disclosed herein. This definition of a human antibody specifically excludes a humanized antibody. A “human antibody” as used herein can be produced using various techniques known in the art, including phage-display libraries (e.g. Hoogenboom and Winter, J. Mol. Biol., 227:381 (1991); Marks et al., J. Mol. Biol., 222:581 (1991), hereby incorporated by reference in its entirety), by methods described in Cole et al., Monoclonal Antibodies and Cancer Therapy, Alan R. Liss, p. 77 (1985) (hereby incorporated by reference in its entirety); Boerner et al., J. Immunol., 147(1):86-95 (1991) (hereby incorporated by reference in its entirety), van Dijk and van de Winkel, Curr. Opin. Pharmacol., 5: 368-74 (2001) (hereby incorporated by reference in its entirety), and by administering the antigen (e.g. SEB) to a transgenic animal that has been modified to produce such antibodies in response to antigenic challenge, but whose endogenous loci have been disabled, e.g., immunized xenomice (see, e.g., U.S. Pat. Nos. 5,939,598; 6,075,181; 6,114,598; 6,150,584 and 6,162,963 to Kucherlapati et al. regarding XENOMOUSE™ technology, each of which patents are hereby incorporated by reference in their entirety), e.g. Veloclmmune® (Regeneron, Tarrytown, N.Y.), e.g. UltiMab® platform (Medarex, now Bristol Myers Squibb, Princeton, N.J.). See also, for example, Li et al., Proc. Natl. Acad. Sci. USA, 103:3557-3562 (2006) regarding human antibodies generated via a human B-cell hybridoma technology. See also KM Mouse® system, described in PCT Publication WO 02/43478 by Ishida et al., in which the mouse carries a human heavy chain transchromosome and a human light chain transgene, and the TC mouse system, described in Tomizuka et al. (2000) Proc. Natl. Acad. Sci. USA 97:722-727, in which the mouse carries both a human heavy chain transchromosome and a human light chain transchromosome, both of which are hereby incorporated by reference in their entirety. In each of these systems, the transgenes and/or transchromosomes carried by the mice comprise human immunoglobulin variable and constant region sequences.

The term “human antibody”, as used herein, is intended to include antibodies having variable regions in which both the framework and CDR regions are sequences of human origin or identical thereto. Furthermore, if the antibody (e.g. an intact antibody rather than, for example, an Fab fragment) contains a constant region, the constant region also is derived from such human sequences, e.g., human germline sequences, or mutated versions of human germline sequences. The human antibodies of the invention may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. In one non-limiting embodiment, where the human antibodies are human monoclonal antibodies, such antibodies can be produced by a hybridoma which includes a B cell obtained from a transgenic nonhuman animal, e.g., a transgenic mouse, having a genome comprising a human heavy chain transgene and a light chain transgene fused to an immortalized cell. In addition, the term “human antibody” as used herein specifically excludes an antibody produced in a human.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region (HVR) of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin variable domain are replaced by corresponding non-human residues. These modifications may be made to further refine antibody performance. Furthermore, in a specific embodiment, humanized antibodies may comprise residues that are not found in the recipient antibody or in the donor antibody. In an embodiment, the humanized antibodies do not comprise residues that are not found in the recipient antibody or in the donor antibody. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. See, e.g., Jones et al., Nature 321:522-525 (1986); Riechmann et al., Nature 332:323-329 (1988); Presta, Curr. Op. Struct. Biol. 2:593-596 (1992); Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409, the contents of each of which references and patents are hereby incorporated by reference in their entirety. In one embodiment where the humanized antibodies do comprise residues that are not found in the recipient antibody or in the donor antibody, the Fc regions of the antibodies are modified as described in WO 99/58572, the content of which is hereby incorporated by reference in its entirety.

Techniques to humanize a monoclonal antibody are described in U.S. Pat. Nos. 4,816,567; 5,807,715; 5,866,692; 6,331,415; 5,530,101; 5,693,761; 5,693,762; 5,585,089; and 6,180,370, the content of each of which is hereby incorporated by reference in its entirety.

A number of “humanized” antibody molecules comprising an antigen-binding site derived from a non-human immunoglobulin have been described, including antibodies having rodent or modified rodent V regions and their associated complementarity determining regions (CDRs) fused to human constant domains. See, for example, Winter et al. Nature 349: 293-299 (1991), Lobuglio et al. Proc. Nat. Acad. Sci. USA 86: 4220-4224 (1989), Shaw et al. J. Immunol. 138: 4534-4538 (1987), and Brown et al. Cancer Res. 47: 3577-3583 (1987), the content of each of which is hereby incorporated by reference in its entirety. Other references describe rodent hypervariable regions or CDRs grafted into a human supporting framework region (FR) prior to fusion with an appropriate human antibody constant domain. See, for example, Riechmann et al. Nature 332: 323-327 (1988), Verhoeyen et al. Science 239: 1534-1536 (1988), and Jones et al. Nature 321: 522-525 (1986), the content of each of which is hereby incorporated by reference in its entirety. Another reference describes rodent CDRs supported by recombinantly veneered rodent framework regions—European Patent Publication No. 0519596 (incorporated by reference in its entirety). These “humanized” molecules are designed to minimize unwanted immunological response toward rodent anti-human antibody molecules which limits the duration and effectiveness of therapeutic applications of those moieties in human recipients. The antibody constant region can be engineered such that it is immunologically inert (e.g., does not trigger complement lysis). See, e.g. PCT Publication No. WO99/58572; UK Patent Application No. 9809951.8. Other methods of humanizing antibodies that may also be utilized are disclosed by Daugherty et al., Nucl. Acids Res. 19: 2471-2476 (1991) and in U.S. Pat. Nos. 6,180,377; 6,054,297; 5,997,867; 5,866,692; 6,210,671; and 6,350,861; and in PCT Publication No. WO 01/27160 (each incorporated by reference in their entirety).

Other forms of humanized antibodies have one or more CDRs (CDR L1, CDR L2, CDR L3, CDR H1, CDR H2, or CDR H3) which are altered with respect to the original antibody, which are also termed one or more CDRs “derived from” one or more CDRs from the original antibody.

In embodiments, the antibodies or fragments herein can be produced recombinantly, for example antibodies expressed using a recombinant expression vector transfected into a host cell, antibodies isolated from a recombinant, combinatorial human antibody library, antibodies isolated from an animal (e.g., a mouse) that is transgenic for human immunoglobulin genes.

As used herein, the terms “is capable of specifically binding”, “specifically binds”, or “preferentially binds” refers to the property of an antibody or fragment of binding to the (specified) antigen with a dissociation constant that is <1 μM, preferably <1 nM and most preferably <10 pM. In an embodiment, the K_{d} of the antibody for SEB is 250-500 pM. An epitope that “specifically binds”, or “preferentially binds” (used interchangeably herein) to an antibody or a polypeptide is a term well understood in the art, and methods to determine such specific or preferential binding are also well known in the art. A molecular entity is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to an SEB conformational epitope is an antibody that binds this epitope with greater affinity, avidity, more readily, and/or with greater duration than it binds to other SEB epitopes or non-SEB epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding.

The term “compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

Depending on the amino acid sequences of the constant domains of their heavy chains, antibodies (immunoglobulins) can be assigned to different classes. The antibody or fragment can be, e.g., any of an IgG, IgD, IgE, IgA or IgM antibody or fragment thereof, respectively. In an embodiment the antibody is an immunoglobulin G. In an embodiment the antibody fragment is a fragment of an immunoglobulin G. In an embodiment the antibody is an IgG1, IgG2, IgG2a, IgG2b, IgG3 or IgG4. In an embodiment the antibody comprises sequences from a human IgG1, human IgG2, human IgG2a, human IgG2b, human IgG3 or human IgG4. A combination of any of these antibodies subtypes can also be used. One consideration in selecting the type of antibody to be used is the desired serum half-life of the antibody. For example, an IgG generally has a serum half-life of 23 days, IgA 6 days, IgM 5 days, IgD 3 days, and IgE 2 days. (Abbas A K, Lichtman A H, Pober J S. Cellular and Molecular Immunology, 4th edition, W.B. Saunders Co., Philadelphia, 2000, hereby incorporated by reference in its entirety).

In an embodiment the antibody or fragment neutralizes SEB when bound thereto. In an embodiment the antibody or fragment does not neutralize SEB when bound thereto alone, but does neutralize SEB when bound thereto with another antibody.

The “variable region” or “variable domain” of an antibody refers to the amino-terminal domains of the heavy or light chain of the antibody. The variable domain of the heavy chain may be referred to as “V_{H}.” The variable domain of the light chain may be referred to as “V_{L}.” These domains are generally the most variable parts of an antibody and contain the antigen-binding sites. The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any vertebrate species can be assigned to one of two clearly distinct types, called kappa (κ) and lambda (λ) based on the amino acid sequences of their constant domains.

“Framework” or “FR” residues are those variable domain residues other than the HVR residues as herein defined.

The term “hypervariable region” or “HVR” when used herein refers to the regions of an antibody variable domain which are hypervariable in sequence and/or form structurally defined loops. Generally, antibodies comprise six HVRs; three in the V_{H} (H1, H2, H3) and three in the V_{L} (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996). A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991) hereby incorporated by reference in its entirety). Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the V_{H}. The variable domain residues are numbered according to Kabat et al., supra, for each of these definitions.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine of the Fc region may be removed, for example, during production or purification of the antibody, or by recombinantly engineering the nucleic acid encoding a heavy chain of the antibody. Accordingly, an intact antibody as used herein may be an antibody with or without the otherwise C-terminal cysteine.

As used herein a “conformational epitope” of SEB is an epitope formed by a plurality of amino acids, at least two of which are discontinuous, arranged in a three-dimensional conformation due to the native folding of the antigen. The conformational epitope is recognized by the antigen-binding portion of an antibody directed to the conformational epitope.

Compositions or pharmaceutical compositions comprising the antibodies, ScFvs or fragments of antibodies disclosed herein are preferably comprise stabilizers to prevent loss of activity or structural integrity of the protein due to the effects of denaturation, oxidation or aggregation over a period of time during storage and transportation prior to use. The compositions or pharmaceutical compositions can comprise one or more of any combination of salts, surfactants, pH and tonicity agents such as sugars can contribute to overcoming aggregation problems. Where a composition or pharmaceutical composition of the present invention is used as an injection, it is desirable to have a pH value in an approximately neutral pH range, it is also advantageous to minimize surfactant levels to avoid bubbles in the formulation which are detrimental for injection into subjects. In an embodiment, the composition or pharmaceutical composition is in liquid form and stably supports high concentrations of bioactive antibody in solution and is suitable for parenteral administration, including intravenous, intramuscular, intraperitoneal, intradermal and/or subcutaneous injection. In an embodiment, the composition or pharmaceutical composition is in liquid form and has minimized risk of bubble formation and anaphylactoid side effects. In an embodiment, the composition or pharmaceutical composition is isotonic. In an embodiment, the composition or pharmaceutical composition has a pH or 6.8 to 7.4.

In an embodiment the ScFvs or fragments of antibodies disclosed herein are lyophilized and/or freeze dried and are reconstituted for use.

Examples of pharmaceutically acceptable carriers include, but are not limited to, phosphate buffered saline solution, sterile water (including water for injection USP), emulsions such as oil/water emulsion, and various types of wetting agents. Preferred diluents for aerosol or parenteral administration are phosphate buffered saline or normal (0.9%) saline, for example 0.9% sodium chloride solution, USP. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000, the content of each of which is hereby incorporated in its entirety). In non-limiting examples, the can comprise one or more of dibasic sodium phosphate, potassium chloride, monobasic potassium phosphate, polysorbate 80 (e.g. 2-[2-[3,5-bis(2-hydroxyethoxy)oxolan-2-yl]-2-(2-hydroxyethoxy)ethoxy]ethyl (E)-octadec-9-enoate), disodium edetate dehydrate, sucrose, monobasic sodium phosphate monohydrate, and dibasic sodium phosphate dihydrate.

The antibodies, or fragments of antibodies, or compositions, or pharmaceutical compositions described herein can also be lyophilized or provided in any suitable forms including, but not limited to, injectable solutions or inhalable solutions, gel forms and tablet forms.

The term “K_{d}”, as used herein, is intended to refer to the dissociation constant of an antibody-antigen interaction. One way of determining the K_{d} or binding affinity of antibodies to SEB is by measuring binding affinity of monofunctional Fab fragments of the antibody. (The affinity constant is the inverted dissociation constant). To obtain monofunctional Fab fragments, an antibody (for example, IgG) can be cleaved with papain or expressed recombinantly. The affinity of an anti-SEB Fab fragment of an antibody can be determined by surface plasmon resonance (BIAcore3000™ surface plasmon resonance (SPR) system, BIAcore Inc., Piscataway N.J.). CM5 chips can be activated with N-ethyl-N′-(3-dimethylaminopropyl)-carbodiinide hydrochloride (EDC) and N-hydroxysuccinimide (NHS) according to the supplier's instructions. SEB can be diluted into 10 mM sodium acetate pH 4.0 and injected over the activated chip at a concentration of 0.005 mg/mL. Using variable flow time across the individual chip channels, two ranges of antigen density can be achieved: 100-200 response units (RU) for detailed kinetic studies and 500-600 RU for screening assays. Serial dilutions (0.1-10× estimated K_{d}) of purified Fab samples are injected for 1 min at 100 microliters/min and dissociation times of up to 2 h are allowed. The concentrations of the Fab proteins are determined by ELISA and/or SDS-PAGE electrophoresis using a Fab of known concentration (as determined by amino acid analysis) as a standard. Kinetic association rates (kₒₙ) and dissociation rates (k_{off}) are obtained simultaneously by fitting the data to a 1:1 Langmuir binding model (Karlsson, R. Roos, H. Fagerstam, L. Petersson, B. (1994). Methods Enzymology 6, 99-110, the content of which is hereby incorporated in its entirety) using the BIA evaluation program. Equilibrium dissociation constant (K_{d}) values are calculated as k_{off}/kₒₙ. This protocol is suitable for use in determining binding affinity of an antibody or fragment to any SEB. Other protocols known in the art may also be used. For example, ELISA of SEB with mAb can be used to determine the k_{D} values. The K_{d} values reported herein used this ELISA-based protocol.

As used herein, the term “subject” for purposes of treatment includes any subject, and preferably is a subject who is in need of the treatment of the targeted pathologic condition for example an SEB-associated pathology. For purposes of prevention, the subject is any subject, and preferably is a subject that is at risk for, or is predisposed to, developing the targeted pathologic condition for example SEB-associated pathology. As used herein, “prevent” means attenuating the development or establishment of, or attenuating the extent of, the disease in the relevant subject. The term “subject” is intended to include living organisms, e.g., prokaryotes and eukaryotes. Examples of subjects include mammals, e.g., humans, dogs, cows, horses, pigs, sheep, goats, cats, mice, rabbits, rats, and transgenic non-human animals. In specific embodiments of the invention, the subject is a human.

As used herein a “small molecule” is an organic compound either synthesized in the laboratory or found in nature which contains carbon-carbon bonds, and has a molecular weight of less than 2000. In an embodiment, the small molecule has a molecular weight of less than 1500. The small molecule may be a substituted hydrocarbon or an substituted hydrocarbon.

In an embodiment, the SEB has the sequence:

| (SEQ ID NO: 1) | |
|---|---|
| ESQPDPKPDE LHKSSKFTGL MENMKVLYDD NHVSAINVKS IDQFLYFDLI YSIKDTKLGN | 60 |
| YDNVRVEFKN KDLADKYKDK YVDVFGANYY YQCYFSKKTN DINSHQTDKR KTCMYGGVTE | 120 |
| HNGNQLDKYR SITVRVFEDG KNLLSFDVQT NKKKVTAQEL DYLTRHYLVK NKKLYEFNNS | 180 |
| PYETGYIKFI ENENSFWYDM MPAPGDKFDQ SKYLMMYNDN KMVDSKDVKI EVYLTTKKK. | 239 |

In an embodiment, the SEB modified to remove the C-terminal 11 residues has the sequence:

| (SEQ ID NO: 2) | |
|---|---|
| ESQPDPKPDE LHKSSKFTGL MENMKVLYDD NHVSAINVKS IDQFLYFDLI YSIKDTKLGN | 60 |
| YDNVRVEFKN KDLADKYKDK YVDVFGANYY YQCYFSKKTN DINSHQTDKR KTCMYGGVTE | 120 |
| HNGNQLDKYR SITVRVFEDG KNLLSFDVQT NKKKVTAQEL DYLTRHYLVK NKKLYEFNNS | 180 |
| PYETGYIKFI ENENSFWYDM MPAPGDKFDQ SKYLMMYNDN KMVDSKDV | 228 |

In an embodiment, the SEB from MRSA has the sequence:

| (SEQ ID NO: 3) | |
|---|---|
| ESQPDPKPDE LHKSSKFTGL MENMKVLYDD NHVSAINVKS IDQFLYFDLI YSIKDTKLGN | 60 |
| YDNVRVEFKN KDLADKYKDK YVDVFGANYY YQCYFSKKTN DINSHQTDKR KTCMYGGVTE | 120 |
| HNGNQLDKYR SITVRVFEDG KNLLSFDVQT NKKKVTAQEL DYLTRHYLVK NKKLYEFNNS | 180 |
| PYETGYIKFI ENENSFWYDM MPAPGDKFDQ SKYLMMYNDN KMVDSKDVKI EVYLYDKEK | 239 |

In an embodiment, the SEA has the sequence:

| (SEQ ID NO: 4) | |
|---|---|
| SEKSEEINEK DLRKKSELQG TALGNLKQIY YYNEKAKTEN KESHDQFLQH TILFKGFFTD | 60 |
| HSWYNDLLVD FDSKDIVDKY KGKKVDLYGA YYGYQCAGGT PNKTACMYGG VTLHDNNRLT | 120 |
| EEKKVPINLW LDGKQNTVPL ETVKTNKKNV TVQELDLQAR RYLQEKYNLY NSDVFDGKVQ | 180 |
| RGLIVFHTST EPSVNYDLFG AQGQYSNTLL RIYRDNKTIN SENMHIDIYL YTS | 233 |

In an embodiment, TSST-1 has the sequence:

| (SEQ ID NO: 5) | |
|---|---|
| STNDNIKDLL DWYSSGSDTF TNSEVLDNSL GSMRIKNTDG SISLIIFPSP YYSPAFTKGE | 60 |
| KVDLNTKRTK KSQHTSEGTY IHFQISGVTN TEKLPTPIEL PLKVKVHGKD SPLKYGPKFD | 120 |
| KKQLAISTLD FEIRHQLTQI HGLYRSSDKT GGYWKITMND GSTYQSDLSK KFEYNTEKPP | 180 |
| INIDEIKTIE AEIN | 194 |

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Introduction

Herein the generation and characterization of monoclonal antibodies (mAbs) to SEB is described. Their toxin neutralizing efficacy in two murine models of SEBILS is also demonstrated. Site-directed mutagenesis provides new insight into the conformational epitope target, and neutralization studies in animal models highlight ways to decrease dose and improve efficacy of anti-SEB antibody therapies.

### Materials and Methods

S. aureus Toxins

The toxins SEA, SEB, and TSST-1 were purchased from Toxin Technology (Sarasota, Fla.) in accordance with CDC biosafety regulations. Recombinant full-length SEB and SEB deletion mutants were generated in compliance with 42 C.F.R. Parts 72, 73, and health and safety regulations. The commercially available SEB toxin is derived from a methicillin sensitive S. aureus strain (MSSA).

mAbs

mAbs to SEB were generated from SEB-immunized BALB/c mice in the Hybridoma Facility of Albert Einstein College of Medicine (AECOM) as described. All mice were immunized with full-length SEB (MSSA derived) in complete Freund adjuvans (CFA). The mouse with the highest Ab titer to SEB was selected for spleen harvest and hybridoma generation. Hybridoma supernatants were screened for reactivity to SEB by ELISA, with positive reactivity being defined as absorbance 3-fold higher than background. Four mAbs, 20B1, 14G8, 6D3, and 4C7 were selected and used in this study. Specificity of mAb for SEB was determined by Western blot according to standard methods with purified SEA, SEB, and TSST-1.

T-Cell Proliferation and Cytokine Assays

T-cells were isolated from donor blood using RosetteSep CD4+ T-cell enrichment mixture (Stemcell Tech) and T-cell proliferation was measured using the ViaLight HS Cell Proliferation kit (Cambrex BioScience), both according to manufacturer's instructions. Briefly, T-cells (5×10⁴/well) were stimulated in 96 well cultureplates with 100 μM of purified SEB (Toxin Technology). SEB-specific mAbs (500 nM) were added concurrently with SEB. Cells were incubated at 37° C. with 10% CO₂ for 48 and 96 h. Next, 100 μl per well of nucleotide releasing reagent was added and incubated for 10 min to lyse cells followed by 20 μl of ATP monitoring reagent. The plates were immediately read with is integrated read times. For cytokine induction assays, purified T-cells were mixed 1:1 with donor matched PBMCs. Supernatants were removed after 8 h of co-incubation with SEB and mAbs and measured by ELISA for human IL-2 and IFN-γ (21).

Sequence Analysis of Variable (V) Region of mAb

RNA was isolated from hybridoma culture cells with a Qiagen RNeasy kit and cDNA was prepared using Superscript II (Invitrogen). Amplification of variable regions was done by PCR using previously published primers (22). The resulting amplification products were gel purified and sequenced in both directions using M13 primers. Sequence was analyzed using BLAST 2 sequence and amino acid sequence was generated using the program “Translate” from ExPASy proteomic server. The sequences obtained for heavy and light chain V regions were further analyzed for homologous germline variable region genes in the database using IMGT (International ImMuno-GeneTics Information System) software program. The AID generated SHM of Immunoglobulin variable (V) regions was analyzed by SHM tool webserver (23).

Sequence Analysis and Deletion Mutation Analysis

Sequence analysis of SEB gene in clinical MSSA and methicillin-resistant (MRSA) S. aureus isolates was performed by isolating DNA by Qiagen DNeasy blood and tissue kit (Qiagen) according to manufacturer's instructions. PCR amplification of the SEB gene was done using specific primers (SEB-for 5′-GAGAGTCAACCAGATCCTAA-3′ (SEQ ID NO:6) and SEB-rev 5′-GCAGGTACTCTATAAGTGCCTGC-3′ (SEQ ID NO:7)). Purified PCR products were ligated into TOPO-TA cloning vector (Invitrogen) and transformed in Top-10 E. coli competent cells and purified by standard methods for sequencing. Sequences were aligned in ClustalW with SEB gene sequence of S. aureus (M11118).

Purification of SEB

Full-length SEB gene from MRSA and MSSA encoding the residues 1-239 and SEB deletion mutants 1-7 were subcloned into H-MBP-T vector (24) using the primers shown below (SEQ ID NOs: 12-17, respectively):

SEB-for:
5′-GTAGCAGGATCCGAGAGTCAACCAGATCCTAAACC-3′
SEB-rev:
5′-CATCGTGTCGACTCACTTTTTCTTTGTCGTAAGATAAAC-3′
SEB-MRSA-rev:
5′-CATCGTGTCGACTCATTTTTCTTTGTCGTAAAGATA AAC-3′
SEB mutant-1-rev:
5′-CATCGTGTCGACTCAAAGATAAACTTCAATCTTCACATC-3′
SEB mutant-2-rev:
5′-CATCGTGTCGACTCACACATCTTTAGAATCAACCATTTT-3′
SEB mutant-3-rev:
5′-CATCGTGTCGACTCAATCAACCATTTTATTGTCATTGTA-3′
SEB mutant-4-rev:
5′-CATCGTGTCGACTCAGTCAAATTTATCTCCTGGTGCAGG-3′
SEB mutant-5-rev:
5′-CATCGTGTCGACTCAAAATTTAATATATCCCGTTTCATA-3′
SEB mutant-6-rev:
5′-CATCGTGTCGACTCATTGTACGTCAAAAGATAATAAATT-3′
SEB mutant-7-for:
5′-GTAGCAGGATCCTACTTTGACTTAATATATTCTATT-3′

H-MBP-TSEB plasmid was then transformed into Escherichia coli BL-21(DE3) Codon Plus (Stratagene) cells for protein expression. Cells were grown for ˜18 h at 15° C. in LB media after inducing with 0.5 mM IPTG at 0.6 OD. Cells were harvested and re-suspended in 20 mM Tris, pH 7.5 and lysed with 1× Bug-Buster. The clear supernatant was incubated with 5 ml of Talon affinity resin (Clontech) for 1 h. The resin was washed with the lysis buffer and the fusion protein was eluted with the lysis buffer supplemented with 200 mM imidazole. The eluted protein was digested with thrombin overnight at 4° C. to cleave the H-MBP fusion tag and the excess imidazole was removed by dialysis into 20 mM Tris, pH 7.5. The fusion tag and other impurities were removed by using a HiTrap Q Sepharose ion-exchange column (GE HealthCare). The fractions, which contained SEB, were pooled and passed through a size exclusion column pre-equilibrated with buffer (20 mM Tris, pH 7.5) to remove high molecular weight soluble aggregates. The protein was found to be >99% pure by SDS-PAGE. Similarly, all other deletion mutants were cloned into H-MBP-T vector and expressed and purified as mentioned above. Full-length SEB, mutant-1 and mutant-2 proteins were successfully expressed as soluble fraction, however mutants 3-7 expressed as insoluble fraction.

Amino Acid Substitutions of SEB by Site-Directed Mutagenesis

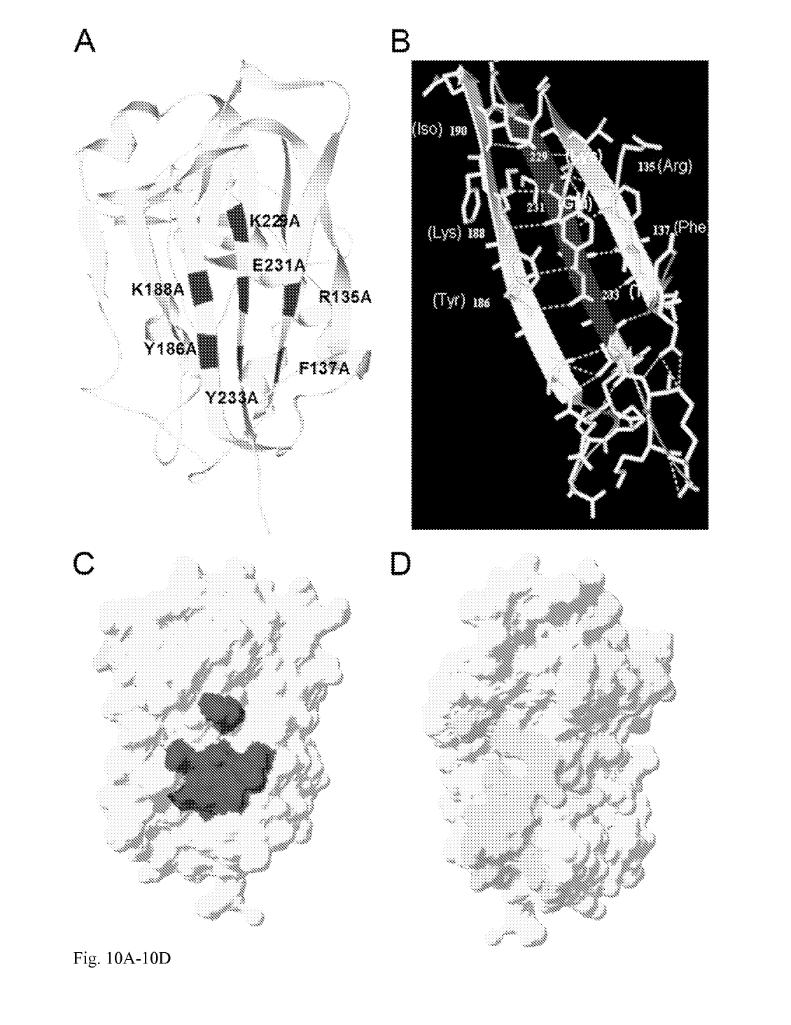

Selected amino acids residues on SEB were mutated by site-directed mutagenesis using Quickchange XL Site-directed Mutagenesis kit (Stratagene, La Jolla, Calif.). Based on computer assisted modeling, we gave precedence to positions where the residues are hydrogen bonded between the backbone C-terminal residues. FIGS. 10A and 10B shows the expanded view of the β-sheet formed by the three strands. To avoid disrupting the overall folding of SEB, 7 AA positions were mutated to alanine, 135-Arg, 137-Phe, 186-Tyr, 188-Lys, 229-Lys, 23′-Glu, 233-Tyr. We also generated mutant-MRSA by adding an extra residue (T) at base position 703. PCR primers were designed using QuickChange® Primer Design Program and PCR was conducted according to manufacturer's instructions. Purified PCR products of mutated clones were ligated into H-MBP-T vector and transformed into Escherichia coli XL-10 gold cells. Substitution of amino acids in all mutant constructs was confirmed by sequencing. Expression and purification of mutant SEBs were done as described above.

SDS-PAGE and Western Blotting

The crude induced and un-induced lysates of SEB, mutant 1-3 and single point mutation proteins were dissolved in 30 μl of sample loading buffer and boiled for 10 min. After centrifugation for 30 s, the proteins were resolved on a 10% SDS-polyacrylamide gel under denaturing conditions and stained with Coomassie Brilliant Blue R-250. For immunoblotting, the proteins were separated on a 10% SDS-polyacrylamide gel, and the fractionated proteins were transferred from the gel onto the PVDF membrane (Millipore) in a semi-dry transblot apparatus. The membrane was blocked in blocking buffer (1×PBS, 0.05% Tween 20, 5% milk) for 2 h. The blots were washed and incubated with 1:20,000 dilution of 10 μg/μl concentration mAbs (20B1 or 14G8 or 6D3) for 45 min. Later, the blots were washed twice in PBST and one in PBS and further incubated for 45 min with HRP (horseradish peroxidase)-conjugated antimouse IgG (1:10,000). After washing, development was performed by chemiluminescence method according to manufacturer's instructions (Thermo Scientific). Further binding to mutant proteins and C-terminal decapeptide were investigated under native conditions using dot blot analysis. Briefly 2 μg of synthesized 10-mer peptide (Genscript Corporation), SEB and the mutant-1 and 2 protein were spotted onto the nitrocellulose membrane and dried for 10 min. Membranes were further blocked by soaking in blocking buffer for 2 h. Membranes were washed with PBST twice and incubated with 1:10,000 dilution of 10 μg/μl concentration mAbs (20B1 or 14G8 or 6D3) for 45 min. Blots were further washed with PBST twice and incubated with HRP-conjugated anti-mouse IgG1 (1:10,000) and developed as before.

ELISA

Standard ELISA to measure SEB concentration was performed as described (21). To establish relative affinity of mAbs decreasing levels of mAb (0.1-0.001 μg) as well as decreasing levels of SEB toxin (0.1 and 0.001 μg) were used in ELISA assay. ELISA was performed with WT-SEB and purified SEB mutants protein (1 and 2) and point mutation proteins by coating the plate with purified protein, followed by unlabeled mAbs 20B1 or 14G8 or 6D3 or 4C7, which further binds to AP-conjugated anti-mouse IgG1 and was developed by PNPP tablets. A modified competition ELISA was done to determine if two mAbs could bind to SEB simultaneously. This assay involved coating the plate with anti-IgG1 Ab, followed by unlabeled SEB specific mAb (mAbs 20B1 or 6D3 or 14G8 or 4C7) and SEB Ag. After washing another mAb (mAbs 14G8 or 6D3 or 20B1 or 4C7) was added and incubated for 1 h and further captured with a labeled anti-mouse IgG1. Alternatively, this ELISA was also performed with directly labeled mAbs.

An ELISA-based protocol using the mAbs with SEB was used to determine Kₐ values of mAbs. The following affinity Kₐ values were determined:

### 20B1(IgG1)—305.4 pM;

### 14G8(IgG1)—357.733 pM; and

### 6D3 (IgG1)—355.533 pM.

Animal Experiments

All animal experiments were carried out with the approval of the Animal Institute Committee (AIC), in accordance with the rules and regulations set forth by the AECOM AIC. Protective efficacy of mAbs was tested in 2 murine models for SEBILS. BALB/c mice, injected intraperitoneal with 25 mg of D-galactosamine in PBS, followed by 20 μg of purified SEB (Toxin technology) die with 48 h. Transgenic mice expressing HLA-DR3 in the absence of endogenous MHC class II (a generous gift of Dr. David Chella, Mayo Clinic) were injected intraperitoneal with two doses of 50 μg of SEB 48 h apart and die within 4-5 days. To test protective efficacy, mice were injected intraperitoneal once with different doses of mAbs 20B1, 14G8, 6D3, and 4C7, or in combinations 10 min prior to administration of SEB. Control mice were treated with PBS, isotype-specific mAb 18B7 or NSO ascites, which was made by injecting mice with the myeloma cell partner NSO and thus provides an ascites control without specific antibodies. Murine blood was obtained from retro-orbital bleeding at 2, 8, and 24 h post-toxin injection according to animal institute guidelines as outlined by AIC. Serum was separated by centrifugation from clotted blood at 3000 rpm×10 min and frozen prior to measurement by ELISA.

### Results

Generation of mAbs to SEB

All mice immunized with full length SEB (MSSA-derived) in CFA responded to immunization. Eleven hybridomas were successfully stabilized after two soft agar cloning steps that allowed selection for efficient Ab producers with strong binding to SEB. To identify good candidates that could be further developed as potential therapeutic reagents, hybridomas were characterized for isotype and protective efficacy in vivo in BALB/c mice co-injected with SEB and D-galactosamine (Table 1). D-Galactosamine potentiates the SEB effect in mice, which by nature are resistant to SEB. These experiments identified 3 mAbs that conveyed protection, 5 mAbs that conveyed partial protection and 3 mAbs that exhibited no protection against SEBILS. Four IgG1 mAbs (20B1, 6D3, 14G8, and 4C7) were focused on which showed different degrees of protection. Their respective hybridomas had good in vitro growth parameters. Furthermore, IgGs have a long serum half-life time, which makes them suitable candidates for in vivo application.

TABLE 1
List of SEB-specific mAbs and their efficacy
to protect against SEBILS in vivo

| | | Protection in vivo |
|---|---|---|
| mAB | Isotype | (BALB/c) |
| 20B1 | IgG1 | 100% |
| 6D3 | IgG1 | 40-60% |
| 3B4 | IgM | 100% |
| 10F1 | IgA | 100% |
| 14G8 | IgG1 | 0% |
| 14B9 | IgG2a | 60% |
| 11B4 | IgG2a | 60% |
| 17C12 | IgG2a | 60% |
| 4D4 | IgG1 | 20% |
| 12A1 | IgG1 | 20% |
| 4C7 | IgG1 | 0% |

Characterization of mAbs to SEB: Specificity

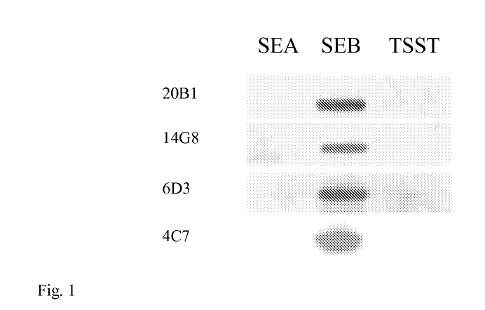

Specificity of mAbs for SEB was evaluated by their binding to SEA, SEB, and TSST-1. Western blot analysis showed that mAbs 20B1, 14G8, 4C7, and 6D3 bound to SEB but not to SEA or TSST-1 (FIG. 1).

SEB Sequence from Clinical Isolates

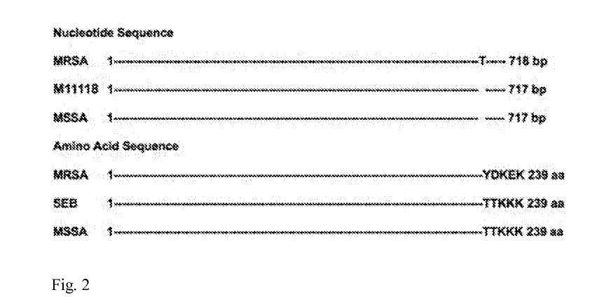

Sequence analysis of SEB genes derived from 9 MRSA and 3 MSSA clinical isolates was performed and compared with the SEB sequence of MSSA strain M11118. An additional nucleotide was found at position 703 in all MRSA but not in any MSSA strain. This addition results in three amino acid changes at positions 235, 236, and 238 (tyrosine-threonine, asparagine-threonine, and glutamine-lysine) (FIG. 2). Multilocus sequence typing (MLST) and spa typing assigned all 9 MRSA isolates to CC8 spa7 type whereas the MSSA strains were assigned to CC5 spa2, CC8 spa 139, and CC8 spa7 type.

Ig Gene Utilization

The germ line genes encoding 3 of the 4 mAbs are shown in Table 2.

| | | | | | V_{L} | | |
|---|---|---|---|---|---|---|---|
| mAb | V_{H} gene | V_{H} family | J_{H} gene | D gene | family | V_{L} gene | J_{L} gene |
| 20B1 | AJ972403 | IGHV9-4*02 | IGHJ4*01 | IGHD2- | IGKV9- | AF003294 | IGKJ1*01 |
| | | | | 1*01 | 124*01 | | |
| 14G8 | X03399 | IGHV5S4*01F | IGHJ3*01 | IGHD2- | IGKV5- | AJ235964 | IGKJ2*01 |
| | | | | 13*01 | 39*01 | | |
| 6D3 | X00160 | IGHV1- | IGHJ4*01 | IGHD3- | IGKV8- | Y15980 | IGKJ5*01 |
| | | 69*02 | | 3*01 | 19*01 | | |

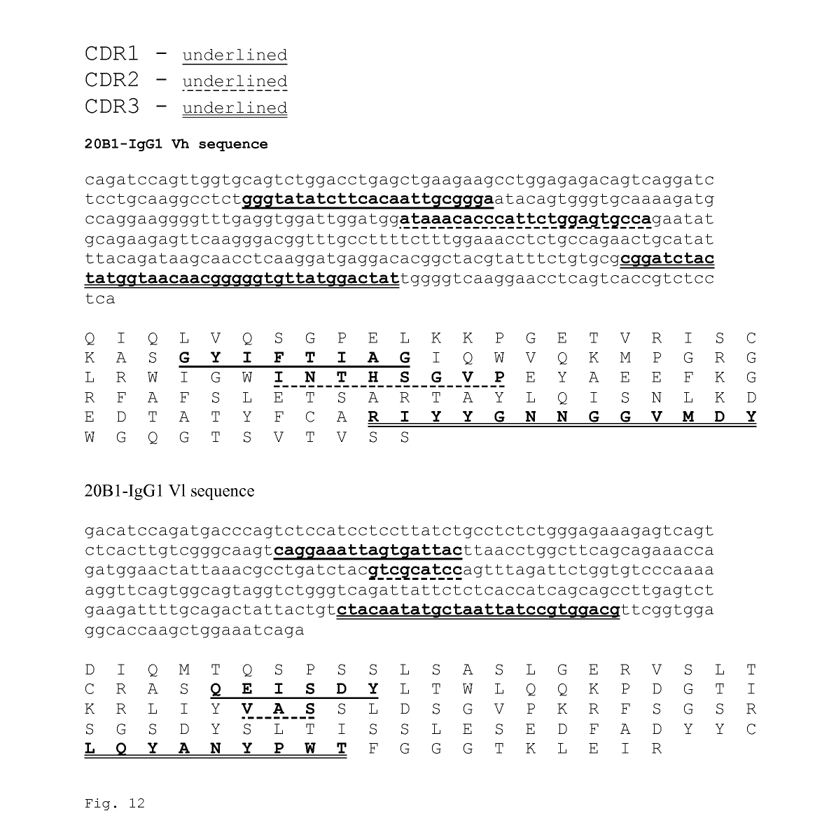

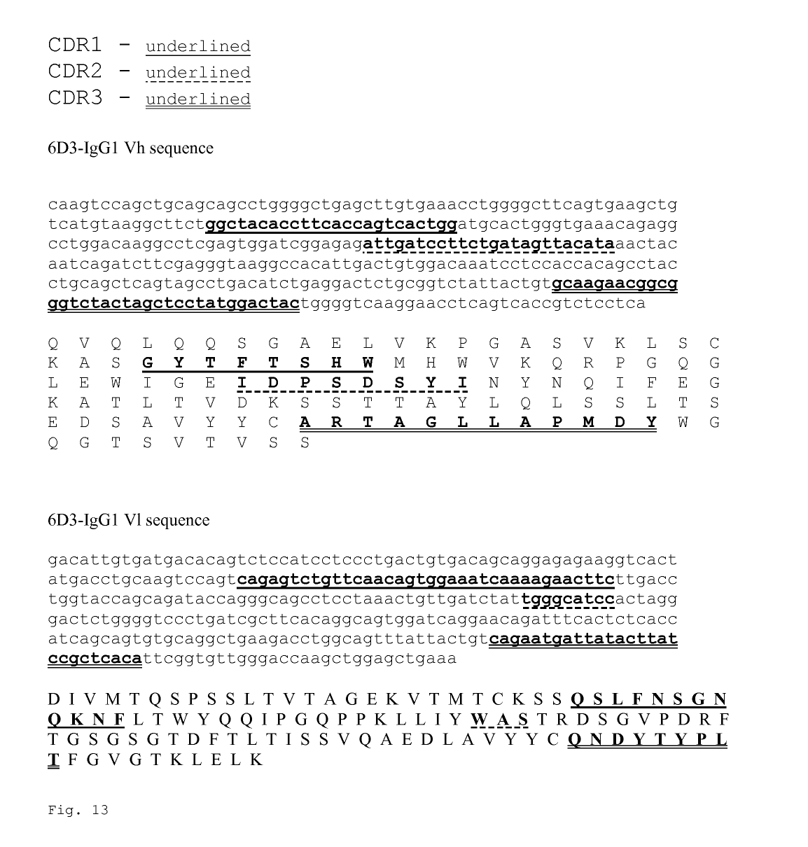

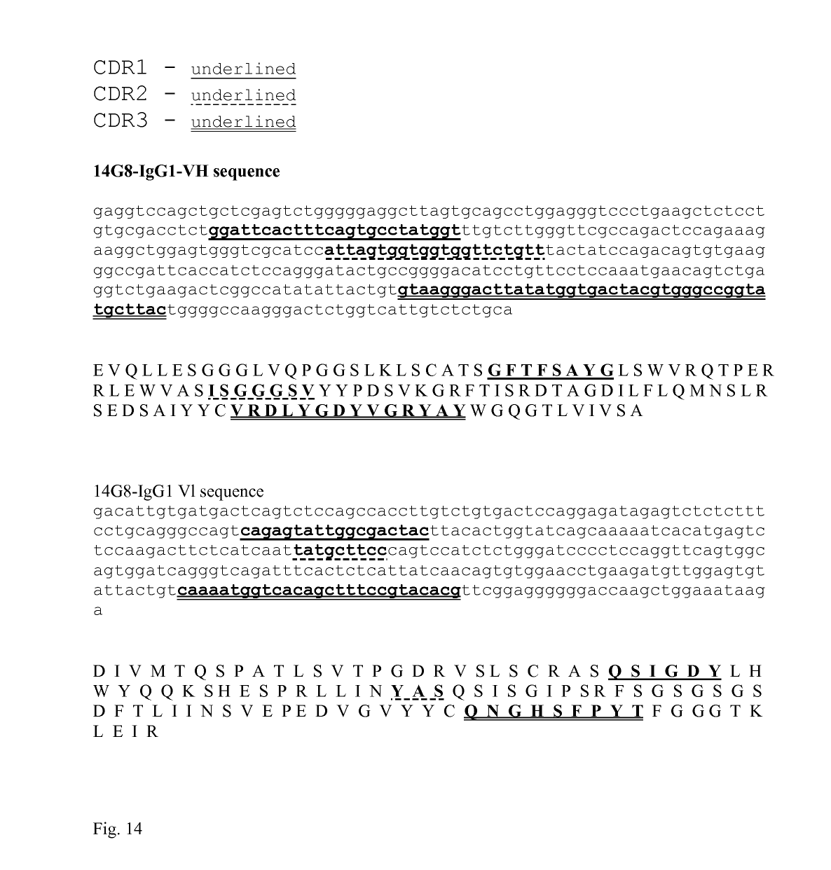

These data demonstrate that each of the 3 mAbs studied were different. The probable CDR regions for these three antibodies are shown in FIGS. 12-14.

TABLE 3A
Percentage of mutations located in
AID and Pol η associated hotspots

| | | | 20B1-VH | 14G8-VH | 6D3-VH |
|---|---|---|---|---|---|
| | | | (10 mutations) | (18 mutations) | (14 mutations) |
| AID | Hot- | WRC | 3 (30%) | 3 (16.67%) | 4 (28.57%) |
| | spot | GYW | 1 (10%) | 5 (27.8%) | 3 (21.42%) |
| | Cold- | SYC | 0 | 1 (5.55%) | 0 |
| | spot | GRS | 0 | 1 (5.55%) | 0 |
| Pol n | | WA | 5 (50%) | 8 (44.4%) | 4 (28.57%) |
| Hotspot | | TW | 1 (10%) | 0 | 3 (21.42%) |

TABLE 3B
Percentage of mutations located in
AID and Pol η associated hotspots

| | | | 20B1-VL | 14G8-VL | 6D3-VL |
|---|---|---|---|---|---|
| | | | (3 mutations) | (8 mutations) | (7 mutations) |
| AID | Hot- | WRC | 0 | 1 (12.5%) | 1 (14.28%) |
| | spot | GYW | 3 (100%) | 2 (25%) | 2 (28.57%) |
| | Cold- | SYC | 0 | 1 (12.5%) | 0 |
| | spot | GRS | 0 | 0 | 0 |
| Pol n | | WA | 0 | 4 (50%) | 4 (57.14%) |
| Hotspot | | TW | 0 | 0 | 0 |

Inhibition of T-cell Proliferation and Cytokine Induction with SEB-specific mAbs

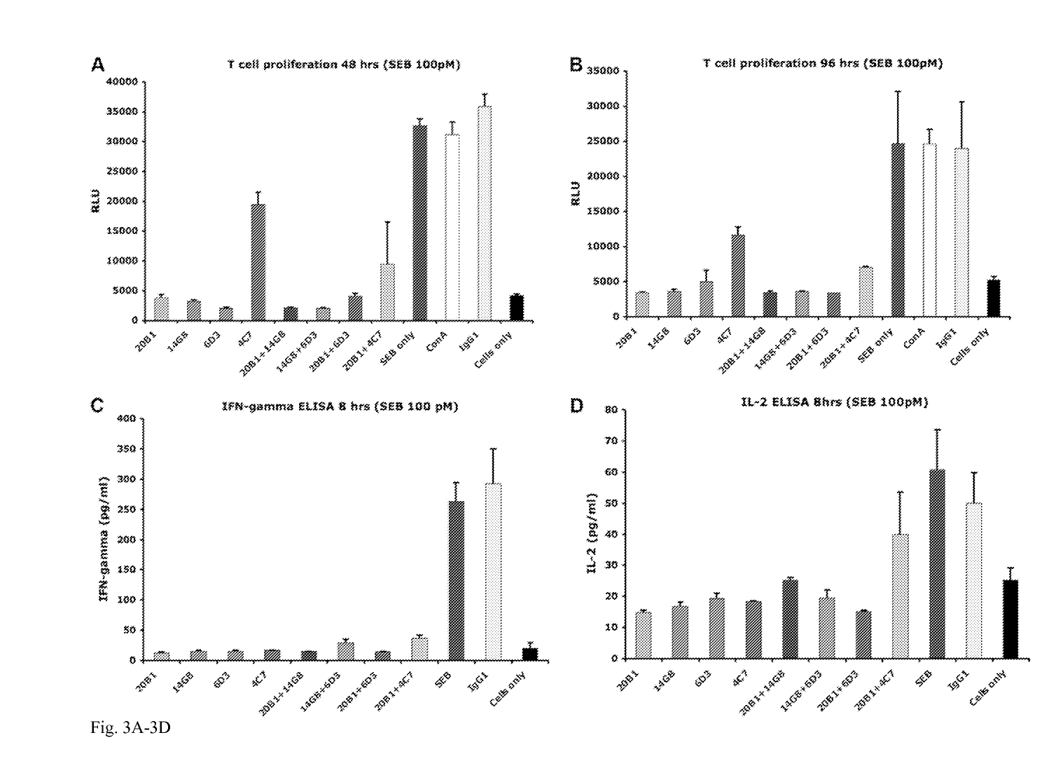

SEB acts as a potent T-cell mitogen that binds to the Vβ chain of the TCR and induces T-cell proliferation and cytokine production. Because the human MHC-II complex has the highest affinity for SEB, humans are more sensitive than mice. Therefore, neutralizing efficacy was also tested in vitro in human T-cells. The effect of SEB-specific mAbs alone or in combination on SEB-induced T-cell proliferation and cytokine production in human T-cells from a normal donor was measured. MAbs 20B1, 14G8, and 6D3 each demonstrated comparable levels of inhibition of SEB-induced T-cell proliferation after 48 and 96 h (FIGS. 3A and 3B) whereas the effect of 4C7 treatment was only half that of the positive controls. Inhibition of cytokine induction was also measured after 8 h and as expected T-cells produced less IFN-γ (FIG. 3C) and IL-2 (FIG. 3D) if treated with SEB-specific mAb when compared with untreated T-cells. These assays also demonstrated comparable inhibition of IFN-γ by mAbs including 4C7. Inhibition of IL-2 excretion was less complete and not observed in mAb 4C7-treated T-cells. Enhanced inhibition of T-cell proliferation and IL-2 production could not be shown for when mAbs were used in combination, however mAb 4C7 used in combination with mAb 20B1 lessened the potent neutralizing effect of mAb 20B1.

SEB-Specific mAbs Protect Mice Against SEBILS

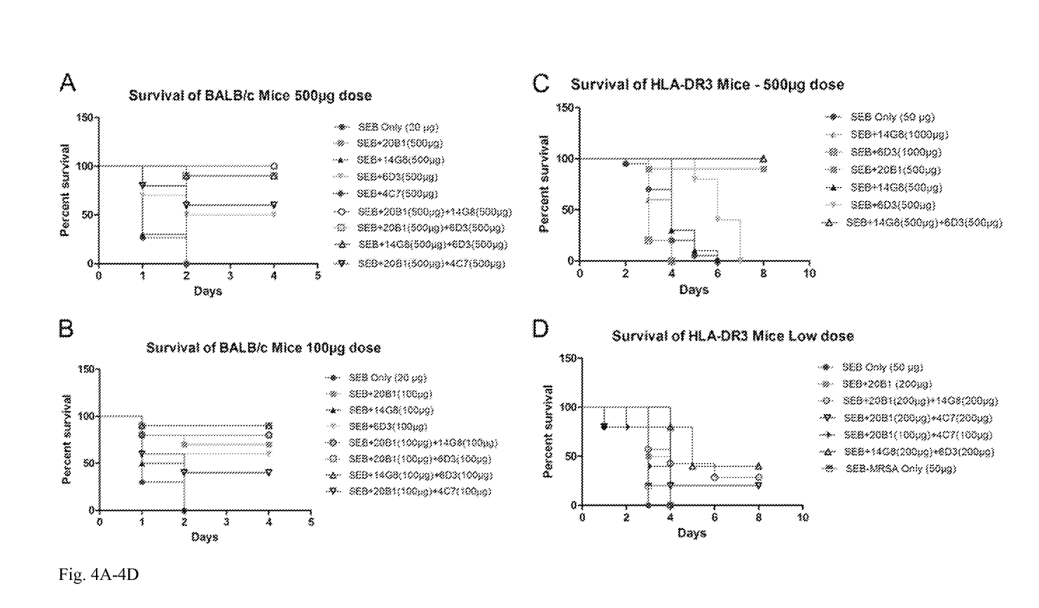

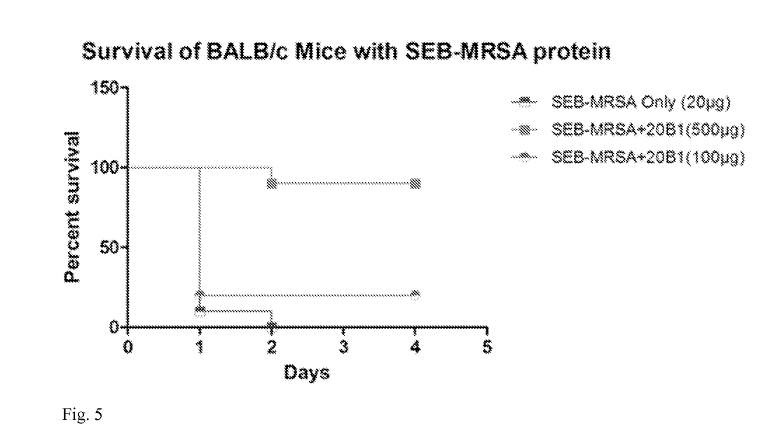

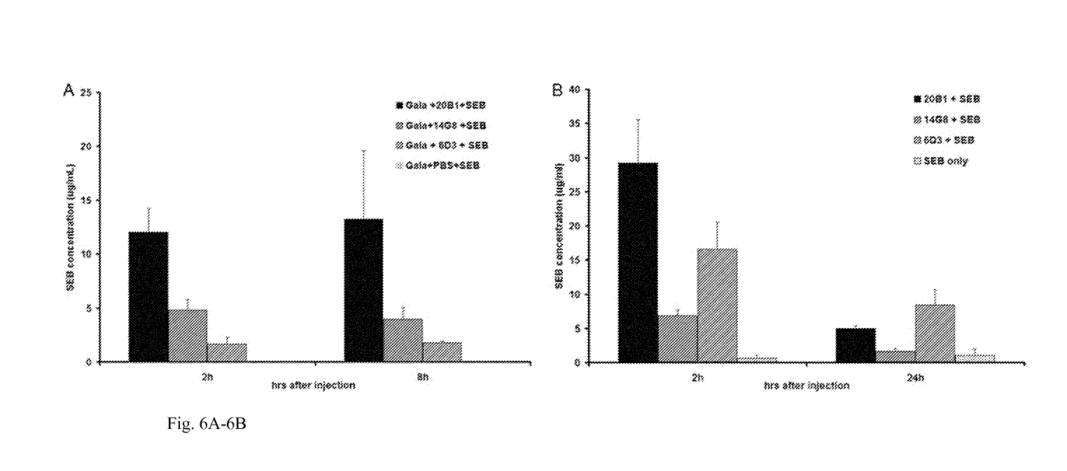

Next, the protective efficacy of mAbs 20B1, 14G8, 6D3 and 4C7 mAbs was explored in vivo in two different models of SEBILS, one in BALB/c and the other in HLA-DR3 transgenic mice. In contrast to in vitro assays, these animal experiments demonstrated significant differences in toxin neutralization for the different mAbs as well as for combinations of mAbs. Protection also differed between the two models. Two of the four mAbs (6D3 and 20B1) demonstrated consistent levels of protection in the D-galactosamine-potentiated BALB/c model (FIG. 4A). Treatment with doses of mAb 20B1 as low as 100 μg per mouse conveyed protection (FIG. 4B). Enhanced protection was observed when mAb 20B1 was given in combination with mAbs 6D3 or 14G8 in doses as low as 50 μg, which were not protective when used as monotherapy. In the BALB/c model 20B1 demonstrated superior efficacy compared with 6D3, which was less protective when used alone in HLA-DR3 (FIG. 4C). MAbs 14G8 and 4C7 treatment did not protect mice from SEBILS in either mouse model. However, mAb 14G8 enhanced protection when used in combination with mAb 20B1 or 6D3 in HLA-DR3 as well as in BALB/c mice whereas 4C7 lowered the efficacy of mAb 20B1 in a manner analogous to that observed for in vitro neutralization assays. In the HLA-DR3 model combination of two non-protective mAbs resulted in 60-100% protection whereas treatment with either one of the mAb could not protect mice from SEBILS (FIG. 4D). Lastly, the protective efficacy of mAbs in mice that were injected with MRSA-derived SEB protein was also investigated. These mice died in the same time frame as those injected with MSSA-derived SEB. Although these mice were protected by treatment with mAbs 20B1, efficacy was decreased as low doses of 100 μg could not convey protection whereas they did when mice were injected with MSSA-derived SEB (FIG. 5). SEB serum levels measured by ELISA were consistently higher in mice (both murine models), treated with mAbs compared with non-treated control mice (FIGS. 6A and 6B). Of note, SEB serum levels in mice correlated with protection. Treatment with one mAb did not interfere with the accurate quantification of SEB in serum but quantification could not be accurately carried out in the setting of combination therapy.

Mapping of SEB-specific Ab Binding Sites

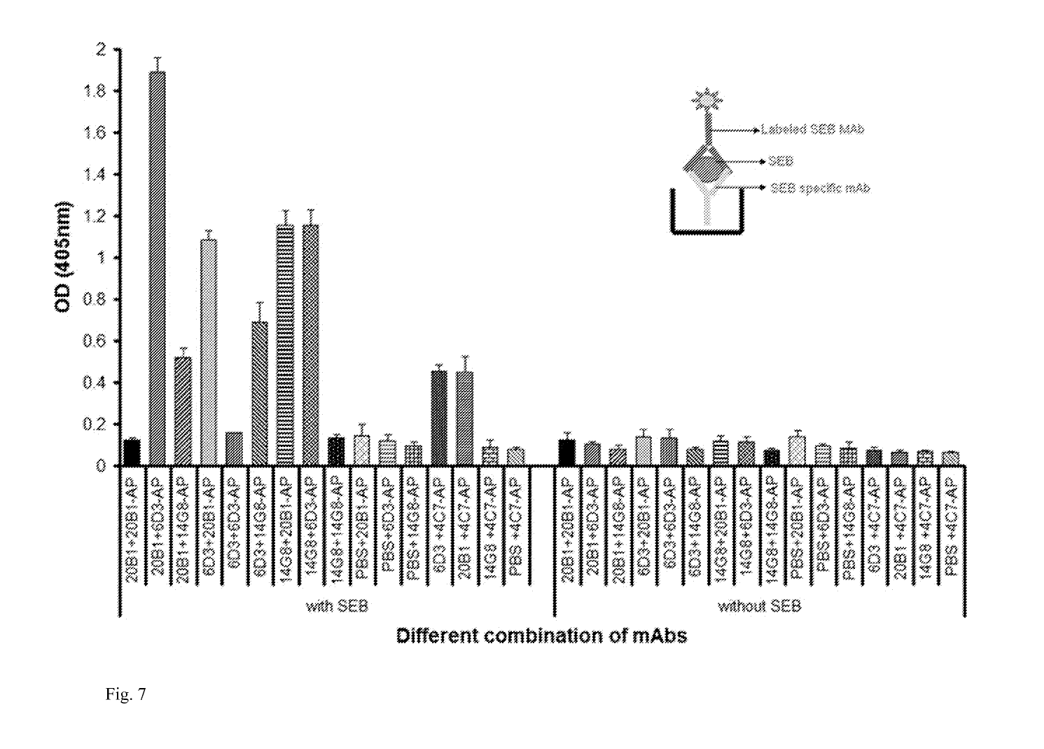

First, the capture ELISA was modified to determine if mAbs recognized distinct epitopes. The results demonstrated that mAbs 20B1, 14G8, and 6D3 each recognized different epitopes and thus can bind in any combination of two of the three mAbs simultaneously (FIG. 7) whereas mAbs 4C7 and 14G8 cannot bind simultaneously.

Also apparent from these experiments was that there is only one relevant epitope present per toxin molecule as binding inhibited additional binding of the same mAb. Competition ELISA where one mAb was kept constant while the other was varied in concentration indicated some concentration-dependent inhibition of binding in the setting of two mAbs (data not shown), which was most significant for mAbs 4C7 and 20B1.

Deletion Mutational Analysis of SEB-Specific mAbs Binding

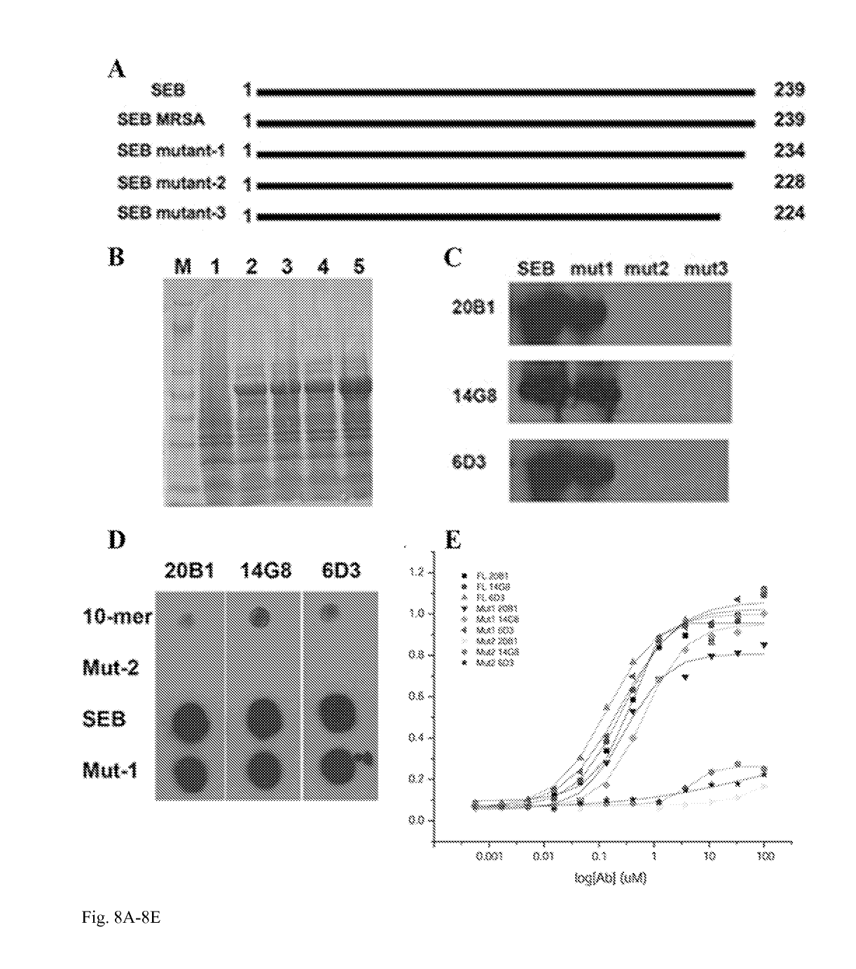

To investigate the domains recognized by the various mAbs to SEB, mutant proteins were cloned in accordance with select agent regulations (42CFR73). Full-length SEB, SEB-MRSA, three C-terminal deletions of 5, 11, 15, residues (mutants 1-3) and mutants of aa 1-209, 1-189, 1-149, 46-149 (mutant 4-7) (FIG. 8A) were successfully expressed. All mAbs recognized the full-length SEB protein, deletion mutant-1 (5 terminal residues deleted) and the MRSA-derived SEB protein (addition of thymidine at 703). Further deletion of the C-terminus (11 and 15 residues) eliminated binding as measured by Western blot (FIG. 8(C)) and ELISA (FIG. 8(E)). Dot blot analysis comparing binding of mAbs to the decapeptide (227-236), SEB and mutant-1 demonstrated binding of the mAbs to the decapeptide (FIG. 8(D)) but not mutant-2, however binding efficiency was variable. Given that the C-terminus distal 10 residue epitope would be too small to accommodate distinct binding of 4 mAbs it was concluded that the actual mAb binding domain was more complex and included conformational epitopes to which distantly located residues contribute. Consequently the C-terminus would be either directly part of several conformational epitopes each binding one of the mAbs or contribute indirectly to their stability.

Site-Directed Mutagenesis

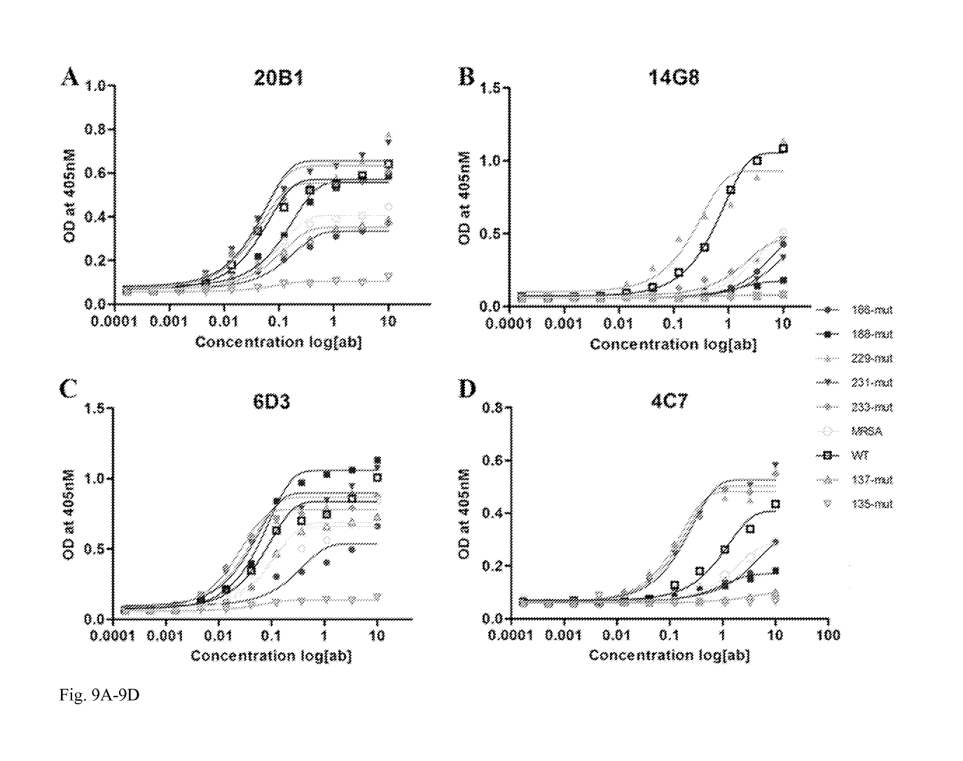

To identify individual amino acids that could be involved in epitope structure, we focused on 7 residues based on computer-assisted three-dimensional modeling derived from crystal structure of SEB (FIG. 10) (2, 25) (Brookhaven Protein Data Bank—accession code 3SEB) that were hydrogen-bonded to the residues of the C-terminus and make up a centrally located β-stranded sheet. The Tyr, Phe, and Lys side chains of these amino acids are solvent exposed and therefore could interact with V region of mAbs. By site directed mutagenesis the residues (135-Arg, 137-Phe, 186-Tyr, 188-Lys, 229-Lys, 231-Glu, 233-Tyr) were replaced by Ala and the binding of mAbs to the mutated proteins, wild type SEB (WT SEB) (SEQ ID NO:1) and MRSA-derived SEB protein was compared by ELISA (FIG. 9).

These assays demonstrated that the binding of the mAbs was differentially affected by site-directed mutagenesis of these residues, with the most common outcome being decreased binding relative to WT SEB. Based on decreases in binding, residues 135-R, 137-F, 186-Y, 235- and 236-T interacted with mAb 20B1 (FIG. 9A), whereas mAb 14G8 interacted with residues 135-R, 137-F, 186-Y, 188-K, 231-E, 233-Y, and 235, 236-T (FIG. 9B). The residues 135-R, 186-Y were required for the interaction with mAb 6D3 (FIG. 9C), and 135-R, 137-F, 186-Y, 188-K, and 235, 236-T were involved in the binding of mAb 4C7 (FIG. 9D). An interesting finding was that the binding of mAb 4C7 was enhanced by certain mutations. Overall, these data also support previous dot blot data that suggested enhanced binding of mAb 14G8 to the decapeptide when compared with mAb 20B1. The latter mAb uses only 235 and 236 residues in the C-terminal whereas mAb 14G8 binds also to residues 231 and 233. Consistent with a difference in neutralizing efficacy evident in animal models of SEBILS, these assays also underscored the differences of MRSA- and MSSA-derived SEB.

S. aureus intravenous (i.v.) model: Pathogenesis of SEB-producing S. aureus infection was explored and the protective efficacy of SEB-specific mAb was tested in vivo using a BALB/c murine model for systemic infection (e.g. a bacteremia, septicemia model). In this model, a suspension of 5×10⁷ SEB-producing MRSA was effective i.v. to kill the mice. An SEB-producing MRSA strain was used. The actual CFU injected was confirmed by plate counts of the inocula.

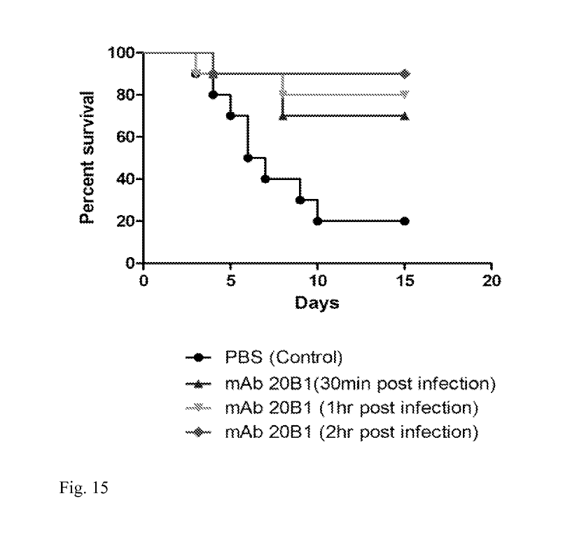

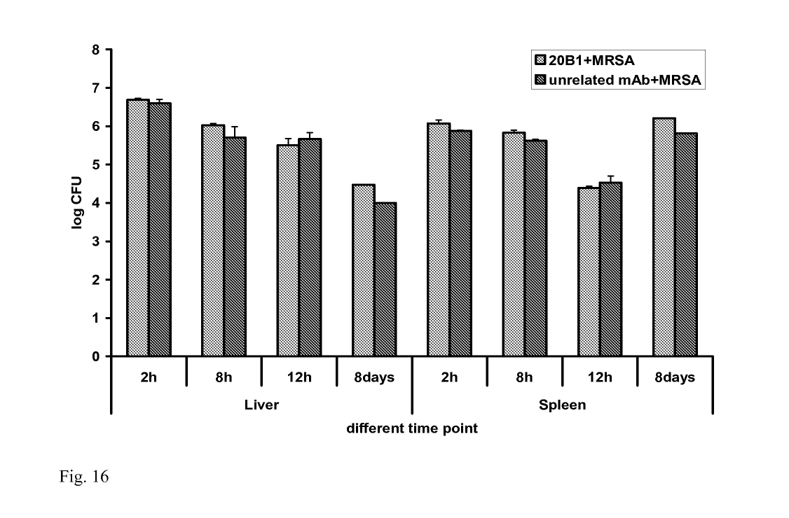

SEB-specific mAb 20B1 (500 μg) or PBS was injected i.v. at different time points (30 min, 1 h and 2 h) after S. aureus infection. Mice were given an i.v. injection of 5×10⁷ of SEB-producing clinical MRSA strains and observed for mortality over 15 days. Clinically infected mice became inactive, huddled together in the cage; and death was observed after the 3rd day post-infection. Mice that underwent treatment with SEB-specific mAb 20B1 survived significantly longer compared to those mice treated with PBS treated mice (FIG. 15) (p=0.003). In different time point experiments (2 h, 8 h, 12 h, and 8 days), the mice were euthanized and liver and spleen excised and organ CFU quantified. There was no difference in S. aureus CFU cultured between liver and spleen from treated or untreated mice at any of the time points (FIG. 16). This further supports that neutralizing SEB mAbs work by counteracting the toxin-mediated inflammatory response, which leads to shock, rather than decreasing pathogen burden. In a patient simultaneous treatment with antibiotics would reduce pathogen burden.

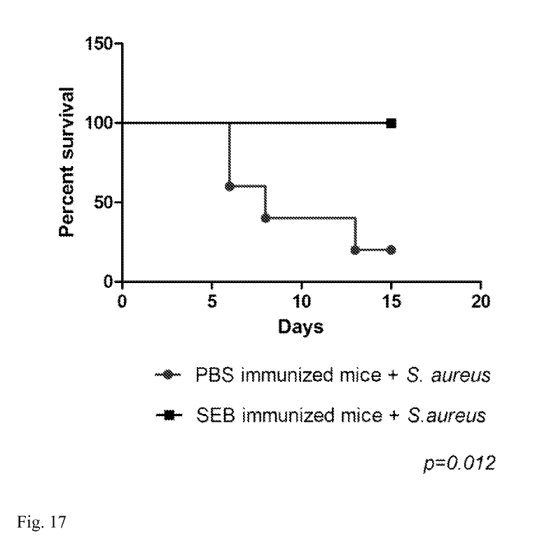

To further support the concept that humoral immune response against SEB is effective protection against a lethal dose of an SEB-producing MRSA, in vivo polyclonal antibodies were generated against SEB toxin by immunizing mice with SEB Immunization was carried out in BALB/c mice by intra-peritoneally injecting SEB protein with CFA followed by a booster dose of SEB protein emulsified in IFA. Control mice were injected with CFA and IFA in PBS according to the immunization schedule. Murine sera were assayed by ELISA to determine titers of SEB-specific mAbs. Titers were >1:100,000 after 22 days. Mice were infected i.v. with an SEB-producing MRSA strain and observed for 15 days. Again, significant survival differences in SEB immunized mice were documented compared to sham immunized mice (FIG. 17) (p=0.012). CFU count in liver and spleen of immunized versus sham immunized mice at 19 days post infection was not affected was equal from both group.

Mouse Skin infection model for S. aureus: SEB-producing MRSA or MSSA strains also commonly cause severe soft tissue infection, for instance in post-surgery patients. BALB/c mice (6-8 weeks old), as a soft tissue infection model, were injected i.p. once with SEB-specific mAb 20B1 (500 ug) or unrelated mAb 24 h prior to S. aureus infection. The hair on the back of mice was shaved and the skin disinfected with ethanol. Single punch biopsies were performed on their backs, resulting in 5 mm diameter full thickness excision wounds. A suspension containing 5×10⁷ of SEB-producing MRSA or non-SEB producing MRSA strains in PBS was inoculated directly onto the wound.

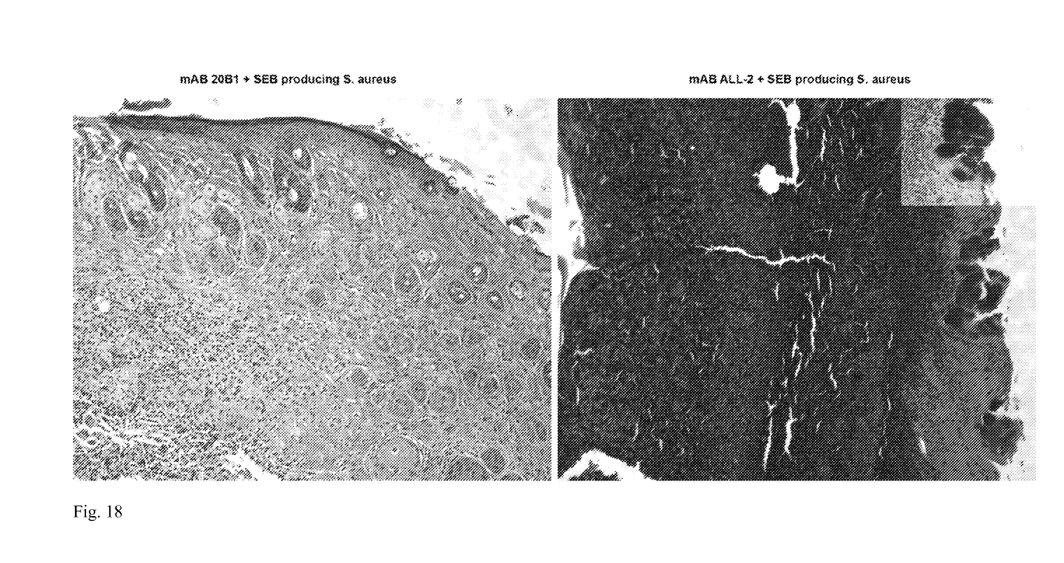

On day 3 and 5, mice were euthanized and wound sections were obtained for histological and CFU analysis. Wound sections were homogenized in PBS and plated onto tryptic soy agar. Excised skin lesion tissues were embedded in paraffin and stained with hematoxylin and eosin, or Gram's stain to observe the morphology, or bacteria, respectively. (FIG. 18).

At day 5, as determined by the size of eschar, mAB 20B1+SEB-producing MRSA was healed compared to the unrelated mAb or the non-SEB producing MRSA strain. The data supports that SEB-specific mAb can change the outcome of infection with SEB-excreting MRSA strains.

### Discussion

The data presented here on four murine mAbs to SEB, which bind to conformational epitopes that are destroyed by deletion of the distal C-terminal 11 amino acids. Three of four mAbs inhibited SEB induced T-cell proliferation as well as IL-2 and IFN-γ production by human T-cells in vitro. However, when tested in murine models these mAbs differed in their protective efficacy against SEBILS. In addition, the data are the first to show that MRSA-derived SEB contains an addition in the C-terminal, which affects binding of certain protective Abs. It is also demonstrated that enhanced protection against SEBILS can be obtained when two “non-protective” mAbs were combined in vivo even if they were not protective in monotherapy. The findings support the concept that mAb combination treatment should be contemplated, even when the individual Abs are not effective as they may be useful in toxin clearance and neutralization when combined.

Several studies have shown that other Abs can be of use against SEBILS in diverse animal models and species (14, 15, 26-29). Although vaccination would be a very effective method to protect humans from toxins, it carries a risk, is costly, and not necessary for all people, as natural immunity could be present and effective (30, 31). Therefore, in recent years major efforts have been undertaken to develop passive immunization therapies against a variety of toxins including potential biological weapons (32). The major advantage of mAbs is that they are biochemically defined reagents that can be readily manufactured in unlimited supply. Although some mAbs have been generated for SEB, most of these studies demonstrate only efficacy or binding in vitro (33-35). In other studies mAbs were generated by vaccination with SEB fragments that recognize the MHC II or Vβ TCR binding site on SEB (13). In our study, we vaccinated mice with MSSA-derived full-length SEB.

In this study mAb protection induced by SEBILS was investigated in two animal models; BALB/c (5, 36) and HLA-DR3. For a number of reasons some studies have proposed that the transgenic HLA-DR3 mouse model is the superior animal model for SEBILS (38-40). In the present study, 100% protection was achieved in both murine models against SEBILS with mAb 20B1. In contrast, mAb 14G8 was not protective and mAb 6D3 was partially protective only in BALB/c mice. No protection was achieved in HLA-DR3 mice administered either only mAb 14G8 or only mAb 6D3, even when using high doses. In contrast, protection was achieved in both murine models when combinations of one protective and one non-protective mAb (20B1 & 14G8 or 20B1 & 6D3) or two “non-protective” mAbs only (14G8 and 6D3) were administered simultaneously even when lower doses were used.

This is the first demonstration of enhanced protection against SEBILS in the BALB/c as well as HLA-DR3 model when two non-protective mAbs (14G8 and 6D3) are combined. Additionally, the experiments with MRSA-derived SEB protein suggest that mAb 20B1 can be used for protection from both MSSA- and MRSA-derived SEB toxicity although higher doses are required for neutralization of MRSA-derived SEB. Previous studies have proposed that the C-terminal residues constitutes the predominant epitope recognized by human polyclonal serum (20). The studies here present a more complex picture. Instead, it is demonstrated that the C terminus constitutes a complex region involved in correct folding of the SEB. Binding studies with the decapeptide indicate that the C-terminal region of SEB may include some linear epitopes (particularly residues 235 and 236 for mAbs 20B1, 14G8, and 4C7), but mostly these residues are critical for maintaining the conformational structure of this region of SEB that is part of a larger conformational epitope. It is evident from the crystal structures that the C-terminal region is well folded and forms an anti-parallel β-sheet as shown in FIG. 10(B) (41). Previous mutational studies have demonstrated that the C-terminal region of SEB does not bind to MHC class II or TCR (3) but is critical for the conformation of the SEB molecule (42). The studies support this conclusion as the loss of the last 11 C-terminal residues result in loss of mAb binding, whereas deleting the last 5 residues did not cause any loss of binding or toxicity. Presumably the conformation of epitopes is disrupted as the deletion of the last 11 residues removes a central strand from the β-sheet, which destabilizes the overall fold of SEB.

Modified capture ELISA in this study demonstrated that 2 mAbs can bind simultaneously to SEB, which would not be expected if the epitope was solely 11 residue long linear sequence. Point mutation SEB clones were generated using site-directed mutagenesis which confirmed that binding of these mAbs is also affected by residues that are not in the linear part of the C-terminal region, but rather interact with the correctly folded C-terminal, thus contributing to more complex conformational epitopes of SEB. Site-directed mutagenesis identified several residues that affect binding of the individual mAbs differentially. It is proposed that two mAbs can bind simultaneously because they bind to secondary and tertiary conformational eptiopes in this region. This finding is relevant because mAbs administered simultaneously confer enhanced protection. Furthermore these assays confirm that each epitope is present only once on a SEB toxin molecule.

The detection of an additional nucleotide at position 703 in the SEB of all clinical MRSA strains tested, and not in MSSA strains, may affect folding and Ab neutralization resulting in biological advantages that promoted its selection. Detection of toxin sequence variation is relevant because it highlights potential mechanisms of evasion of the immune response that have to be taken into consideration when passive immunotherapy and vaccination is designed.

Several Abs that recognize conformational epitopes have been described, such as the mAbs that are employed in diagnosing misfolded prion proteins (43). Without being bound by theory, it is possible that mAb binding to SEB can promote conformational changes of SEB and destabilize the MHC-TCRSEB trimer formation, which is critical to confer toxicity.

Clearance of toxin is an important aspect for successful toxin neutralization assay. Although earlier studies have shown that SEB is excreted renally (44), it is not known if mAb treatment can affect renal clearance. The present study indicates that in experimental SEBILS the SEB serum levels in are consistently higher in mice treated with SEB-specific mAb than in control mice. SEB serum levels differed for the individual mAbs but correlated with protective efficacy. Experiments done 50 years ago with SEB specific polyclonal sera also demonstrated prolonged clearance of SEB in blood of injected monkeys (45). It is counterintuitive to think that prolonged serum life correlates with protection, but binding to SEB by mAbs may induce conformational changes and prevent further interaction with cellular receptors and or renal clearance. This mechanism could be operative even though the MHC class II and TCR binding sites on SEB are distant from the epitope that presumably binds the mAbs. mAbs 14G8 and 6D3 achieved protection to SEBILS in HLA-DR3 mice only when administered in combination and never alone, even at higher doses. Unfortunately SEB levels in mice treated with 2 mAbs cannot be accurately determined as combination of mAbs interfered with the ELISA. Cooperative binding of mAbs may induce conformational changes in the toxin thereby altering affinities (allosteric effect) or promote FCR mediated uptake of the immunocomplex, which could not be investigated with FCR knock-out mice because they exhibit inconsistent sensitivity to SEBILS. In pneumococcal pneumonia, treatment with combination of two protective mAbs also enhanced protection against the devastating effects of pneumolysin (46). Furthermore, investigators have shown that in the treatment of viral diseases including rabies and SARS, combination of mAbs against wild-type epitope and variant epitope can prevent the emergence of escape variants (47, 48). Moreover several studies have shown that targeting more than one adhesion protein with mAb in S. aureus infection can be beneficial (49, 50). The finding that mice were better protected against SEBILS by the combination of protective and non (or less)-protective mAbs may have important implications for current FDA regulations which state that “non- or low protective mAb when used individually, fail to show efficacy would not be further considered even though they may be highly effective when used in combination against a potentially lethal disease.” In the setting of intoxications, toxin clearance could be of pivotal importance and further improved by mutating the Fc portion of mAbs, which would affect Fc R binding and Fc_R-mediated uptake. (see, for example, WO/2006/130834, the content of which is hereby incorporated by reference in its entirety).

### REFERENCES

1. Iandolo, J. J., and Shafer, W. M. (1977) Infect. Immun 16, 610-616
2. Swaminathan, S., Furey, W., Pletcher, J., and Sax, M. (1992) Nature 359, 801-806
3. Kappler, J. W., Herman, A., Clements, J., and Marrack, P. (1992) J. Exp. Med. 175, 387-396
4. Faulkner, L., Cooper, A., Fantino, C., Altmann, D. M., and Sriskandan, S. (2005) J. Immunol. 175, 6870-6877
5. Miethke, T., Wahl, C., Heeg, K., Echtenacher, B., Krammer, P. H., and Wagner, H. (1992) J. Exp. Med. 175, 91-98
6. Bachert, C., Zhang, N., Patou, J., van Zele, T., and Gevaert, P. (2008) Curr. Opin. Allergy Clin. Immunol. 8, 34-38
7. Liu, T., Kong, W., Yang, P., and Wang, B. (2006) J. Huazhong. Univ. Sci. Technolog. Med. Sci. 26, 63-67
8. Rossi, R. E., and Monasterolo, G. (2004) Int. Arch. Allergy Immunol. 133, 261-266
9. Rossi, R. J., Muralimohan, G., Maxwell, J. R., and Vella, A. T. (2004) Int. Immunol. 16, 1751-1760
10. Ulrich, R. G., Sidell, S., Taylor, T. J., Wilhelmsen, C. L., and Franz, D. R. (2001) Textbook of Military Medicine: Medical Aspects of Chemical and Biological Warfare, Borden Institute, Washington D.C.
11. Darenberg, J., Söderquist, B., Normark, B. H., and Norrby-Teglund, A. (2004) Clin Infect. Dis. 38, 836-842
12. Bavari, S., Ulrich, R. G., and LeClaire, R. D. (1999) J. Infect. Dis. 180, 1365-1369
13. Hamad, A. R., Herman, A., Marrack, P., and Kappler, J. W. (1994) J. Exp. Med. 180, 615-621
14. LeClaire, R. D., and Bavari, S. (2001) Antimicrob. Agents Chemother. 45, 460-463
15. LeClaire, R. D., Hunt, R. E., and Bavari, S. (2002) Infect. Immun. 70, 2278-2281
16. Boles, J. W., Pitt, M. L., LeClaire, R. D., Gibbs, P. H., Ulrich, R. G., and Bavari, S. (2003) Vaccine 21, 2791-2796
17. Bavari, S., Dyas, B., and Ulrich, R. G. (1996) J. Infect. Dis. 174, 338-345
18. Ulrich, R. G. (1998) Trends Microbiol. 6, 134
19. Ulrich, R. G., Olson, M. A., and Bavari, S. (1998) Vaccine 16, 1857-1864
20. Nishi, J. I., Kanekura, S., Takei, S., Kitajima, I., Nakajima, T., Wahid, M. R., Masuda, K., Yoshinaga, M., Maruyama, I., and Miyata, K. (1997) J. Immunol. 158, 247-254
21. Cook, E., Wang, X., Robiou, N., and Fries, B. C. (2007) Clin Vaccine Immunol. 14, 1094-1101
22. Dattamajumdar, A. K., Jacobson, D. P., Hood, L. E., and Osman, G. E. (1996) Immunogenetics 43, 141-151
23. MacCarthy, T., Roa, S., Scharff, M. D., and Bergman, A. (2009) DNA Repair 8, 137-141
24. Alexandrov, A., Dutta, K., and Pascal, S. M. (2001) BioTechniques 30, 1194-1198
25. Papageorgiou, A. C., Tranter, H. S., and Acharya, K. R. (1998) J. Mol. Biol. 277, 61-79
26. Stiles, B. G., Garza, A. R., Ulrich, R. G., and Boles, J. W. (2001) Infect. Immun. 69, 2031-2036
27. Arad, G., Hillman, D., Levy, R., and Kaempfer, R. (2004) Immunol. Lett. 91, 141-145
28. Woody, M. A., Krakauer, T., and Stiles, B. G. (1997) Vaccine 15, 133-139
29. DaSilva, L., Welcher, B. C., Ulrich, R. G., Aman, M. J., David, C. S., and Bavari, S. (2002) J. Infect. Dis. 185, 1754-1760
30. Holtfreter, S., and Broker, B. M. (2005) Arch. Immunol. Ther. Exp. 53, 13-27
31. Holtfreter, S., Roschack, K., Eichler, P., Eske, K., Holtfreter, B., Kohler, C., Engelmann, S., Hecker, M., Greinacher, A., and Broker, B. M. (2006) J. Infect. Dis. 193, 1275-1278
32. Casadevall, A. (2002) Nat. Biotechnol. 20, 114
33. Alakhov, V. Y., Kabanov, A. V., Batrakova, E. V., Koromyslova, I. A., Levashov, A. V., and Severin, E. S. (1990) Biotechnol. Appl. Biochem. 12, 94-98
34. Alakhov, V., Klinsky, E., Kolosov, M. I., Maurer-Fogy, I., Moskaleva, E., Sveshnikov, P. G., Pozdnyakova, L. P., Shemchukova, O. B., and Severin, E. S. (1992) Eur. J. Biochem. 209, 823-828
35. Li, H. Y., Yao, Y. M., Shi, Z. G., Dong, N., Yu, Y., Lu, L. R., and Sheng, Z. Y. (2003) Shock 20, 257-263
36. Galanos, C., Freudenberg, M. A., and Reutter, W. (1979) Proc. Natl. Acad. Sci. U.S.A. 76, 5939-5943
37. Rajagopalan, G., Sen, M. M., and David, C. S. (2004) Infect. Immun. 72, 6733-6737
38. Rajagopalan, G., Sen, M. M., Singh, M., Murali, N. S., Nath, K. A., Iijima, K., Kita, H., Leontovich, A. A., Gopinathan, U., Patel, R., and David, C. S. (2006) Shock 25, 647-656
39. Rajagopalan, G., Smart, M. K., Cheng, S., Krco, C. J., Johnson, K. L., and David, C. S. (2003) Tissue Antigens 62, 149-161
40. Cheng, S., Smart, M., Hanson, J., and David, C. S. (2003) J. Autoimmun. 21, 195-199
41. L1, H., Llera, A., Tsuchiya, D., Leder, L., Ysern, X., Schlievert, P. M., Karjalainen, K., and Mariuzza, R. A. (1998) Immunity 9, 807-816
42. Metzroth, B., Marx, T., Linnig, M., and Fleischer, B. (1993) Infect. Immun. 61, 2445-2452
43. Caughey, B. (2003) Nat. Med. 9, 819-820
44. Vabulas, R., Bittlingmaier, R., Heeg, K., Wagner, H., and Miethke, T. (1996) Infect. Immun. 64, 4567-4573
45. Normann, S. J. (1970) J. Immunol. 104, 673-678
46. García-Suárez Mdel, M., Cima-Cabal, M. D., Flórez, N., García, P., Cernuda-Cernuda, R., Astudillo, A., Vázquez, F., De los Toyos, J. R., and Méndez, F. J. (2004) Infect. Immun. 72, 4534-4540
47. Bakker, A. B., Marissen, W. E., Kramer, R. A., Rice, A. B., Weldon, W. C., Niezgoda, M., Hanlon, C. A., Thijsse, S., Backus, H. H., de Kruif, J., Dietzschold, B., Rupprecht, C. E., and Goudsmit, J. (2005) J. Virol. 79, 9062-9068
48. Goudsmit, J., Marissen, W. E., Weldon, W. C., Niezgoda, M., Hanlon, C. A., Rice, A. B., Kruif, J., Dietzschold, B., Bakker, A. B., and Rupprecht, C. E. (2006) J. Infect. Dis. 193, 796-801
49. Arrecubieta, C., Matsunaga, I., Asai, T., Naka, Y., Deng, M. C., and Lowy, F. D. (2008) J. Infect. Dis. 198, 571-575
50. Welsch, J. A., Ram, S., Koeberling, O., and Granoff, D. M. (2008) J. Infect. Dis. 197, 1053-1061.

1. An isolated antibody, or an isolated antigen-binding fragment of an antibody, which antibody or antigen-binding fragment binds to staphylococcal enterotoxin B (SEB) and which antibody or antigen-binding fragment comprises a heavy chain variable CDR3 comprising the sequence RIYYGNNGGVMDY (SEQ ID NO:30); ARTAGLLAPMDY (SEQ ID NO:31); ARDTMRKCYCELKLKPPAEHPGPA (SEQ ID NO:32) or VRDLYGDYVGRYAY (SEQ ID NO:48).

2. The antibody or the antigen-binding fragment of claim 1, comprising two heavy chain variable CDR3s each comprising the sequence RIYYGNNGGVMDY (SEQ ID NO:30); ARTAGLLAPMDY (SEQ ID NO:31); ARDTMRKCYCELKLKPPAEHPGPA (SEQ ID NO:32); or VRDLYGDYVGRYAY (SEQ ID NO:48).

3. (canceled)

4. The antibody or the antigen-binding fragment of claim 2, comprising two light chain variable CDR3s each comprising the sequence LQYANYPWT (SEQ ID NO:33); QNDYTYPLT (SEQ ID NO:34); or QNGHSFPYT (SEQ ID NO:35).

5-6. (canceled)

7. The antibody or the antigen-binding fragment of claim 4, comprising two heavy chain variable CDR1s each comprising the sequence GYIFTIAG (SEQ ID NO:36); GYTFTSHW (SEQ ID NO:37); GFTFSSYG (SEQ ID NO:38); or GFTFSAYG (SEQ ID NO:49).

8. The antibody or the antigen-binding fragment of claim 7, comprising two heavy chain variable CDR2s each comprising the sequence INTHSGVP (SEQ ID NO:39); IDPSDSYI (SEQ ID NO:40); INSNGGST (SEQ ID NO:41); or ISGGGSV (SEQ ID NO:50).

9-10. (canceled)

11. The antibody or the antigen-binding fragment of claim 8, comprising two light chain variable CDR1s each comprising the sequence QEISDY (SEQ ID NO:42); QSLFNSGNQKNF (SEQ ID NO:43); or QSIGDY (SEQ ID NO:44).

12. The antibody or the antigen-binding fragment of claim 11, comprising two light chain variable CDR2s each comprising the sequence VAS (SEQ ID NO:45); WAS (SEQ ID NO:46); or YAS (SEQ ID NO:47).

13-14. (canceled)

15. The antibody or the antigen-binding fragment of claim 1, comprising two heavy chain variable CDR1s each comprising the sequence GYIFTIAG (SEQ ID NO:36), two heavy chain variable CDR2s each comprising the sequence INTHSGVP (SEQ ID NO:39), and two heavy chain variable CDR3s each comprising the sequence RIYYGNNGGVMDY (SEQ ID NO:30).

16. The antibody or the antigen-binding fragment of claim 15, comprising two light chain variable CDR1s each comprising the sequence QEISDY (SEQ ID NO:42), two light chain variable CDR2s each comprising the sequence VAS (SEQ ID NO:45), and two light chain variable CDR3s each comprising the sequence LQYANYPWT (SEQ ID NO:33).

17-24. (canceled)

25. A composition comprising the antibody or antigen-binding fragment of claim 1.

26. The composition of claim 25, comprising two or more antibodies or antigen-binding fragments of claim 1, wherein each of the antibodies or antigen-binding fragments comprise different sequences.

27-33. (canceled)

34. An isolated antibody or the antigen-binding fragment of an antibody of claim 1, wherein the antibody is a human antibody, a humanized antibody or a chimeric antibody.

35-49. (canceled)

50. A method of treating a disease associated with a staphylococcus infection in a subject having the disease, or preventing a disease associated with a staphylococcus infection in a subject at risk of the disease, comprising administering to the subject an amount of an antibody, or antigen-binding fragment thereof, of claim 1, or an amount of an antibody directed to a conformational epitope of staphylococcal enterotoxin B (SEB) or an antigen-binding fragment of such antibody, effective to treat the disease.

51. The method of claim 50, wherein the SEB comprises SEQ ID NO:1.

52. (canceled)

53. The method of claim 50, wherein at least two different monoclonal antibodies or antigen-binding fragments thereof directed to a conformational epitope of SEB are administered and their amounts combined are effective to treat the disease.

54. The method of claim 50, wherein the disease is sepsis, SEB-mediated shock, a staphylococcus aureus infection, staphylococcus aureus bacteremia, or staphylococcus aureus-associated atopic dermatitis.

55-57. (canceled)

58. The method of claim 54, wherein the staphylococcus aureus is methicillin-sensitive staphylococcus aureus.

59. The method of claim 53, wherein one antibody is neutralizing and the other antibody is not neutralizing.

60. The method of claim 53, wherein both antibodies are neutralizing.

61. The method of claim 53, wherein neither antibody alone is neutralizing.

62-92. (canceled)

93. An isolated antibody, or the isolated antigen-binding fragment of an antibody, which antibody or antigen-binding fragment binds to staphylococcal enterotoxin B (SEB) comprising SEQ ID NO:1 and which antibody or antigen-binding fragment comprises an amino acid sequence comprising three CDRs, each one of which has at least 90% identity to a different heavy chain variable CDR selected from CDR1, CDR2 and CDR3 as set forth in SEQ ID NO:36, 39, and 30, or from CDR1, CDR2 and CDR3 as set forth in SEQ ID NO:37, 40, and 31, or from CDR1, CDR2 and CDR3 as set forth in SEQ ID NO:49, 50, and 48.

94-123. (canceled)


SEQUENCE LISTING (50 sequences)

SEQ ID NO: 1 - protein, 239 aa, Staphylococcus aureus
ESQPDPKPDELHKSSKFTGLMENMKVLYDDNHVSAINVKSIDQFLYFDLIYSIKDTKLGNYDNVRVEFKNKDLADKYKDKYVDVFGANYYYQCYFSKKTNDINSHQTDKRKTCMYGGVTEHNGNQLDKYRSITVRVFEDGKNLLSFDVQTNKKKVTAQELDYLTRHYLVKNKKLYEFNNSPYETGYIKFIENENSFWYDMMPAPGDKFDQSKYLMMYNDNKMVDSKDVKIEVYLTTKKK

SEQ ID NO: 2 - protein, 228 aa, Staphylococcus aureus
ESQPDPKPDELHKSSKFTGLMENMKVLYDDNHVSAINVKSIDQFLYFDLIYSIKDTKLGNYDNVRVEFKNKDLADKYKDKYVDVFGANYYYQCYFSKKTNDINSHQTDKRKTCMYGGVTEHNGNQLDKYRSITVRVFEDGKNLLSFDVQTNKKKVTAQELDYLTRHYLVKNKKLYEFNNSPYETGYIKFIENENSFWYDMMPAPGDKFDQSKYLMMYNDNKMVDSKDV

SEQ ID NO: 3 - protein, 239 aa, Staphylococcus aureus
ESQPDPKPDELHKSSKFTGLMENMKVLYDDNHVSAINVKSIDQFLYFDLIYSIKDTKLGNYDNVRVEFKNKDLADKYKDKYVDVFGANYYYQCYFSKKTNDINSHQTDKRKTCMYGGVTEHNGNQLDKYRSITVRVFEDGKNLLSFDVQTNKKKVTAQELDYLTRHYLVKNKKLYEFNNSPYETGYIKFIENENSFWYDMMPAPGDKFDQSKYLMMYNDNKMVDSKDVKIEVYLYDKEK

SEQ ID NO: 4 - protein, 233 aa, STAPHYLOCOCCAL AUREUS
SEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYTS

SEQ ID NO: 5 - protein, 194 aa, Staphylococcus aureus
STNDNIKDLLDWYSSGSDTFTNSEVLDNSLGSMRIKNTDGSISLIIFPSPYYSPAFTKGEKVDLNTKRTKKSQHTSEGTYIHFQISGVTNTEKLPTPIELPLKVKVHGKDSPLKYGPKFDKKQLAISTLDFEIRHQLTQIHGLYRSSDKTGGYWKITMNDGSTYQSDLSKKFEYNTEKPPINIDEIKTIEAEIN

SEQ ID NO: 6 - DNA, 20 bp, ARTIFICIAL SEQUENCE
  PRIMER FOR STAPHYLOCOCCAL SP.
gagagtcaaccagatcctaa

SEQ ID NO: 7 - DNA, 23 bp, ARTIFICIAL SEQUENCE
  PRIMER FOR STAPHYLOCOCCAL SP.
gcaggtactctataagtgcctgc

SEQ ID NO: 8 - DNA, 35 bp, ARTIFICIAL SEQUENCE
  PRIMER FOR STAPHYLOCOCCAL SP.
gtagcaggatccgagagtcaaccagatcctaaacc

SEQ ID NO: 9 - DNA, 38 bp, ARTIFICIAL SEQUENCE
  PRIMER FOR STAPHYLOCOCCAL SP.
atcgtgtcgactcactttttctttgtcgtaagataaac

SEQ ID NO: 10 - DNA, 39 bp, ARTIFICIAL SEQUENCE
  PRIMER FOR STAPHYLOCOCCAL SP.
catcgtgtcgactcatttttctttgtcgtaaagataaac

SEQ ID NO: 11 - DNA, 39 bp, ARTIFICIAL SEQUENCE
  PRIMER FOR STAPHYLOCOCCAL SP.
catcgtgtcgactcaaagataaacttcaatcttcacatc

SEQ ID NO: 12 - DNA, 38 bp, ARTIFICIAL SEQUENCE
  PRIMER FOR STAPHYLOCOCCAL SP.
atcgtgtcgactcacacatctttagaatcaaccatttt

SEQ ID NO: 13 - DNA, 39 bp, ARTIFICIAL SEQUENCE
  PRIMER FOR STAPHYLOCOCCAL SP.
catcgtgtcgactcaatcaaccattttattgtcattgta

SEQ ID NO: 14 - DNA, 39 bp, ARTIFICIAL SEQUENCE
  PRIMER FOR STAPHYLOCOCCAL SP.
catcgtgtcgactcagtcaaatttatctcctggtgcagg

SEQ ID NO: 15 - DNA, 39 bp, ARTIFICIAL SEQUENCE
  PRIMER FOR STAPHYLOCOCCAL SP.
catcgtgtcgactcaaaatttaatatatcccgtttcata

SEQ ID NO: 16 - DNA, 39 bp, ARTIFICIAL SEQUENCE
  PRIMER FOR STAPHYLOCOCCAL SP.
catcgtgtcgactcattgtacgtcaaaagataataaatt

SEQ ID NO: 17 - DNA, 36 bp, ARTIFICIAL SEQUENCE
  PRIMER FOR STAPHYLOCOCCAL SP.
gtagcaggatcctactttgacttaatatattctatt

SEQ ID NO: 18 - protein, 121 aa, MOUSE SP.
QIQLVQSGPELKKPGETVRISCKASGYIFTIAGIQWVQKMPGRGLRWIGWINTHSGVPEYAEEFKGRFAFSLETSARTAYLQISNLKDEDTATYFCARIYYGNNGGVMDYWGQGTSVTVSS

SEQ ID NO: 19 - protein, 107 aa, MOUSE SP.
DIQMTQSPSSLSASLGERVSLTCRASQEISDYLTWLQQKPDGTIKRLIYVASSLDSGVPKRFSGSRSGSDYSLTISSLESEDFADYYCLQYANYPWTFGGGTKLEIR

SEQ ID NO: 20 - DNA, 363 bp, MOUSE SP.
cagatccagttggtgcagtctggacctgagctgaagaagcctggagagacagtcaggatctcctgcaaggcctctgggtatatcttcacaattgcgggaatacagtgggtgcaaaagatgccaggaaggggtttgaggtggattggatggataaacacccattctggagtgccagaatatgcagaagagttcaagggacggtttgccttttctttggaaacctctgccagaactgcatatttacagataagcaacctcaaggatgaggacacggctacgtatttctgtgcgcggatctactatggtaacaacgggggtgttatggactattggggtcaaggaacctcagtcaccgtctcctca

SEQ ID NO: 21 - DNA, 321 bp, MOUSE SP.
gacatccagatgacccagtctccatcctccttatctgcctctctgggagaaagagtcagtctcacttgtcgggcaagtcaggaaattagtgattacttaacctggcttcagcagaaaccagatggaactattaaacgcctgatctacgtcgcatccagtttagattctggtgtcccaaaaaggttcagtggcagtaggtctgggtcagattattctctcaccatcagcagccttgagtctgaagattttgcagactattactgtctacaatatgctaattatccgtggacgttcggtggaggcaccaagctggaaatcaga

SEQ ID NO: 22 - protein, 119 aa, MOUSE SP.
QVQLQQSGAELVKPGASVKLSCKASGYTFTSHWMHWVKQRPGQGLEWIGEIDPSDSYINYNQIFEGKATLTVDKSSTTAYLQLSSLTSEDSAVYYCARTAGLLAPMDYWGQGTSVTVSS

SEQ ID NO: 23 - protein, 113 aa, MOUSE SP.
DIVMTQSPSSLTVTAGEKVTMTCKSSQSLFNSGNQKNFLTWYQQIPGQPPKLLIYWASTRDSGVPDRFTGSGSGTDFTLTISSVQAEDLAVYYCQNDYTYPLTFGVGTKLELK

SEQ ID NO: 24 - DNA, 357 bp, MOUSE SP.
caagtccagctgcagcagcctggggctgagcttgtgaaacctggggcttcagtgaagctgtcatgtaaggcttctggctacaccttcaccagtcactggatgcactgggtgaaacagaggcctggacaaggcctcgagtggatcggagagattgatccttctgatagttacataaactacaatcagatcttcgagggtaaggccacattgactgtggacaaatcctccaccacagcctacctgcagctcagtagcctgacatctgaggactctgcggtctattactgtgcaagaacggcgggtctactagctcctatggactactggggtcaaggaacctcagtcaccgtctcctca

SEQ ID NO: 25 - DNA, 339 bp, MOUSE SP.
gacattgtgatgacacagtctccatcctccctgactgtgacagcaggagagaaggtcactatgacctgcaagtccagtcagagtctgttcaacagtggaaatcaaaagaacttcttgacctggtaccagcagataccagggcagcctcctaaactgttgatctattgggcatccactagggactctggggtccctgatcgcttcacaggcagtggatcaggaacagatttcactctcaccatcagcagtgtgcaggctgaagacctggcagtttattactgtcagaatgattatacttatccgctcacattcggtgttgggaccaagctggagctgaaa

SEQ ID NO: 26 - protein, 120 aa, MOUSE SP.
EVQLLESGGGLVQPGGSLKLSCATSGFTFSAYGLSWVRQTPERRLEWVASISGGGSVYYPDSVKGRFTISRDTAGDILFLQMNSLRSEDSAIYYCVRDLYGDYVGRYAYWGQGTLVIVSA

SEQ ID NO: 27 - protein, 107 aa, MOUSE SP.
DIVMTQSPATLSVTPGDRVSLSCRASQSIGDYLHWYQQKSHESPRLLINYASQSISGIPSRFSGSGSGSDFTLIINSVEPEDVGVYYCQNGHSFPYTFGGGTKLEIR

SEQ ID NO: 28 - DNA, 360 bp, MOUSE SP.
gaggtccagctgctcgagtctgggggaggcttagtgcagcctggagggtccctgaagctctcctgtgcgacctctggattcactttcagtgcctatggtttgtcttgggttcgccagactccagaaagaaggctggagtgggtcgcatccattagtggtggtggttctgtttactatccagacagtgtgaagggccgattcaccatctccagggatactgccggggacatcctgttcctccaaatgaacagtctgaggtctgaagactcggccatatattactgtgtaagggacttatatggtgactacgtgggccggtatgcttactggggccaagggactctggtcattgtctctgca

SEQ ID NO: 29 - DNA, 321 bp, MOUSE SP.
gacattgtgatgactcagtctccagccaccttgtctgtgactccaggagatagagtctctctttcctgcagggccagtcagagtattggcgactacttacactggtatcagcaaaaatcacatgagtctccaagacttctcatcaattatgcttcccagtccatctctgggatcccctccaggttcagtggcagtggatcagggtcagatttcactctcattatcaacagtgtggaacctgaagatgttggagtgtattactgtcaaaatggtcacagctttccgtacacgttcggaggggggaccaagctggaaataaga

SEQ ID NO: 30 - protein, 13 aa, MOUSE SP.
RIYYGNNGGVMDY

SEQ ID NO: 31 - protein, 12 aa, MOUSE SP.
ARTAGLLAPMDY

SEQ ID NO: 32 - protein, 24 aa, MOUSE SP.
ARDTMRKCYCELKLKPPAEHPGPA

SEQ ID NO: 33 - protein, 9 aa, MOUSE SP.
LQYANYPWT

SEQ ID NO: 34 - protein, 9 aa, MOUSE SP.
QNDYTYPLT

SEQ ID NO: 35 - protein, 9 aa, MOUSE SP.
QNGHSFPYT

SEQ ID NO: 36 - protein, 8 aa, MOUSE SP.
GYIFTIAG

SEQ ID NO: 37 - protein, 8 aa, MOUSE SP.
GYTFTSHW

SEQ ID NO: 38 - protein, 8 aa, MOUSE SP.
GFTFSSYG

SEQ ID NO: 39 - protein, 8 aa, MOUSE SP.
INTHSGVP

SEQ ID NO: 40 - protein, 8 aa, MOUSE SP.
IDPSDSYI

SEQ ID NO: 41 - protein, 8 aa, MOUSE SP.
INSNGGST

SEQ ID NO: 42 - protein, 6 aa, MOUSE SP.
QEISDY

SEQ ID NO: 43 - protein, 12 aa, MOUSE SP.
QSLFNSGNQKNF

SEQ ID NO: 44 - protein, 6 aa, MOUSE SP.
QSIGDY

SEQ ID NO: 45 - protein, 3 aa, MOUSE SP.
VAS

SEQ ID NO: 46 - protein, 3 aa, MOUSE SP.
WAS

SEQ ID NO: 47 - protein, 3 aa, MOUSE SP.
YAS

SEQ ID NO: 48 - protein, 14 aa, MOUSE SP.
VRDLYGDYVGRYAY

SEQ ID NO: 49 - protein, 8 aa, MOUSE SP.
GFTFSAYG

SEQ ID NO: 50 - protein, 7 aa, MOUSE SP.
ISGGGSV